(19) United States
(12) Patent Application Publication
(10) Pub. No.: US 2026/0167696 A1
(43) Pub. Date: Jun. 18, 2026

(54) HYDROXYPROLINE-FREE HYDROLYZED COLLAGEN PEPTIDES AND METHODS OF MAKING AND USING THE SAME

(71) Applicant: Rochal Technologies, LLC, San Antonio, TX (US)

(72) Inventors: Nina BIONDA, Ithaca, NY (US); Rebecca E. McMAHON, San Antonio, TX (US); Bradley J. RODIER, San Antonio, TX (US); Fahimeh TABATABAEI, Boerne, TX (US); Tiffany F. SANCHEZ, Helotes, TX (US); Sergio A. MEDINA, San Antonio, TX (US); Ann Beal SALAMONE, Gainesville, FL (US)

(73) Assignee: Rochal Technologies, LLC, San Antonio, TX (US)

(21) Appl. No.: 19/422,266

(22) Filed: Dec. 16, 2025

Related U.S. Application Data
(60) Provisional application No. 63/734,476, filed on Dec. 16, 2024.
(60) Provisional application No. 63/734,479, filed on Dec. 16, 2024.

(51) Int. Cl. C07K 14/78; A61K 38/00

(52) U.S. Cl. CPC C07K 14/78; A61K 38/00

(57) ABSTRACT

Provided herein are peptides obtained from hydrolyzed collagen and compositions comprising such. The peptides are from 9 to 100, or 19 to 50 amino acids in length. The peptides are unique from peptides obtained from enzymatically hydrolyzed collagen in that the peptides do not include hydroxyproline. Methods of making the hydroxyproline-free peptides obtained from hydrolyzed collagen, and methods of treatment using the hydroxyproline-free peptides obtained from hydrolyzed collagen are also provided.

### RELATED APPLICATIONS

This application claims the benefit of U.S. Provisional Application No. 63/734,476, filed on Dec. 16, 2024, and U.S. Provisional Application No. 63/734,479, filed on Dec. 16, 2024, the entire disclosure of each of which is incorporated herein by reference.

### SEQUENCE ID LISTING

The instant application contains a Sequence Listing which has been submitted electronically in XML format and is hereby incorporated by reference in its entirety. Said XML copy, created on Dec. 15, 2025, is named F6099-03603_SL.xml, and is 34,143 bytes in size.

### FIELD

This invention relates generally to hydrolyzed collagen peptides and compositions comprising said peptides. In particular, the hydrolyzed collagen peptides are hydroxyproline-free. The hydroxyproline-free hydrolyzed collagen peptides are from 9 to 100 amino acids in length and can be used to treat compromised tissue, e.g., a wound.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Hydrolyzed collagen is derived from acid, alkaline, or enzymatic hydrolysis of collagen (molecular weight of about 300 kDa) to a molecular weight of generally about 12 kDa. The source of collagen can be human, bovine, porcine, piscine, ovine, and avian but can include other sources. Hydrolyzed collagen, when dissolved in aqueous media, produces a low viscosity solution but at 60 wt % solids or higher it can form a water-soluble gel. When dried, this hydrolyzed collagen gel forms a brittle coating that is readily water soluble.

Hydrolyzed collagens are water soluble and have high concentrations of oligomers containing hydroxyproline (hydrophilic), proline (hydrophobic) and glycine (hydrophilic), low content of sulfur-containing amino acids, and no tryptophan.

In humans and other mammals compromised tissue such as wound injury triggers an organized complex cascade of cellular and biochemical events that will in most cases result in a healed wound. A typical healed wound is one that restores normal anatomical structure, function, and appearance on cellular, tissue, organ, and organism levels. Wound healing proceeds via a complex process encompassing a number of overlapping phases, including inflammation, epithelialization, angiogenesis and matrix deposition. These processes lead to a mature wound and a certain degree of scar formation. Compromised tissue repair processes mostly occur along a prescribed course and involves keratinocyte mobility and matrix reconstruction involving the paracrine effect of skin cells, cell migration, and matrix re-assembly.

### SUMMARY

This Summary is not intended to be all-inclusive and the inventions described and claimed herein are not limited to or by the features or embodiments identified in this Summary, which is included for purposes of illustration only and not restriction.

Hydrolyzed collagen peptides which do not include hydroxyproline in the peptide sequence (hydroxyproline-free hydrolyzed collagen peptides) have been obtained and their sequences synthesized de novo, and compositions comprising hydrolyzed collagen peptides generally can be enriched with the addition of supplemental amounts of hydroxyproline-free hydrolyzed collagen peptides which can enhance the properties of hydrolyzed collagen peptides without supplement.

The invention generally relates to peptides and compositions comprising said peptides that can be obtained from hydrolyzed collagen or independently synthesized.

In some aspects, this disclosure provides a hydroxyproline-free peptide comprising the sequence:

(SEQ ID NO: 12)
X₁X₂X₃X₄X₅X₆X₇X₈X₉GPX₁₀GPX₁₁GX₁₂X₁₃X₁₄X₁₅X₁₆X₁₇X₁₈X₁₉X₂₀X₂₁X₂₂
X₂₃X₂₄X₂₅

wherein X₁, X₂, X₃, X₄, X₅, X₆, X₇, X₈, X₉, X₁₂, X₁₃, X₁₄, X₁₅, X₁₆, X₁₇, X₁₈, X₁₉, X₂₀, X₂₁, X₂₂, X₂₃, X₂₄, and X₂₅ are independently selected from no amino acid, direct bond, G, K, I, S, D, R, E, T, Q, A, V, and P;
wherein X₁₀ is selected from I, R, Q, P, and A;
wherein X₁₁ is selected from V, S, P, R, A, and I; and
wherein the peptide does not include hydroxyproline, and pharmaceutically acceptable salts thereof.

In some aspects, this disclosure provides a hydroxyproline-free peptide comprising a sequence selected from:

```
  (SEQ ID NO: 10)
GPPGSPGPA
or
  (SEQ ID NO: 11)
PGTSGPPGLAGPPGSPGPQ;
```

and
wherein the peptide does not include hydroxyproline, and pharmaceutically acceptable salts thereof.

In some aspects, the peptide is from 9 to 100 amino acids in length. In some aspects, the peptide is from 10 to 50 amino acids in length. In some aspects, the peptide is from 15 to 50 amino acids in length. In some aspects, the peptide is from 19 to 50 amino acids in length. In some aspects, the peptide is from 20 to 50 amino acids in length. In some aspects, the peptide is from 10 to 30 amino acids in length. In some aspects, the peptide is a length between any of the aforementioned values.

In some aspects, the peptide is less than 100, 95, 90, 85, 80, 75, 70, 65, 60, 55, 50, 45, 40, 35, 30, 25, 20, 15, 10, or 5 amino acid residues of the larger peptide/protein it was derived from. The term “derived from” means the collagen protein which is hydrolyzed to form the subsequent peptide.

In some aspects, X₁ is G, X₂ is K, X₃ is S, X₄ is G, X₅ is D, X₆ is R, X₇ is G, X₈ is E, X₉ is T, X₁₀ is A, X₁₁ is A, X₁₂ is P, X₁₃ is I, X₁₄ is G, X₁₅ is P, X₁₆ is V; and X₁₇, X₁₈, X₁₉, X₂₀, X₂₁, X₂₂, X₂₃, X₂₄, and X₂₅ are no amino acid such that the peptide comprises the sequence:

(SEQ ID NO: 1)
GKSGDRGETGPAGPAGPIGPV

In some aspects, X₁ is G, X₂ is K, X₃ is S, X₄ is G, X₅ is D, X₆ is R, X₇ is G, X₈ is E, X₉ is T, X₁₀ is A, X₁₁ is A, X₁₂ is P, X₁₃ is V, X₁₄ is G, X₁₅ is A, X₁₆ is R; and X₁₇, X₁₈, X₁₉, X₂₀, X₂₁, X₂₂, X₂₃, X₂₄, and X₂₅ are no amino acid such that the peptide comprises the sequence:

(SEQ ID NO: 2)
GKSGDRGETGPAGPAGPIGPVGAR.

In some aspects, X₁, X₂, X₃, X₄, X₅, X₆, X₇, X₈, and X₉ are no amino acid, X₁₀ is A, X₁₁ is I, X₁₂ is P, X₁₃ is V, X₁₄ is G, X₁₅ is A, X₁₆ is R; and X₁₇, X₁₈, X₁₉, X₂₀, X₂₁, X₂₂, X₂₃, X₂₄, and X₂₅ are no amino acid such that the peptide comprises the sequence:

(SEQ ID NO: 3)
GPAGPIGPVGAR

In some aspects, X₁, X₂, X₃, X₄, X₅, X₆, X₇, X₈, X₉, X₁₄, X₁₅, X₁₆, X₁₇, X₁₈, X₁₉, X₂₀, X₂₁, X₂₂, X₂₃, X₂₄, and X₂₅ are no amino acid, X₁₀ is I, X₁₁ is V, X₁₂ is A, and X₁₃ is R such that the peptide comprises the sequence:

(SEQ ID NO: 4)
GPIGPVGAR

In some aspects, X₁, X₂, X₃, X₄, X₅, X₆, are no amino acid, X₇ is G, X₈ is E, X₉ is G, X₁₀ is O, X₁₁ is R, X₁₂ is S, X₁₃ is E, X₁₄ is G, X₁₅ is P, X₁₆ is Q, X₁₇ is V, X₁₈ is R and X₁₉, X₂₀, X₂₁, X₂₂, X₂₃, X₂₄, and X₂₅ are no amino acid such that the peptide comprises the sequence:

(SEQ ID NO: 5)
GEGGPQGPRGSEGPQGVR

In some aspects, X₁, X₂, and X₃ are no amino acid; X₄ is G, X₅ is P, X₆ is R, X₇ is G, X₈ is P, X₉ is A, X₁₀ is S, X₁₁ is A, X₁₂ is K, X₁₃ is D, X₁₄ is G, X₁₅ is R, X₁₆ is I, X₁₇ is G, X₁₈ is Q, X₁₉ is P, X₂₀ is G, X₂₁ is A, X₂₂ is V, X₂₃ is G, X₂₄ is P, and X₂₅ is A such that the peptide comprises the sequence:

(SEQ ID NO: 6)
GPRGPAGPSGPAGKDGRIGQPGAVGPA

In some aspects, X₁, X₂, X₃, X₄, X₅, X₆, are no amino acid, X₇ is G, X₈ is A, X₉ is V, X₁₀ is R, X₁₁ is S, X₁₂ is P, X₁₃ is Q, and X₁₄, X₁₅, X₁₆, X₁₇, X₁₈, X₁₉, X₂₀, X₂₁, X₂₂, X₂₃, X₂₄, and X₂₅ are no amino acid such that the peptide comprises the sequence:

(SEQ ID NO: 7)
GAVGPRGPSGPQ

In some aspects, X₁, X₂, X₃, X₄, X₅, X₆, are no amino acid, X₇ is G, X₈ is A, X₉ is V, X₁₀ is R, Xu is S, X₁₂ is P, X₁₃ is Q, X₁₄, X₁₅, X₁₆, X₁₇, X₁₈, X₁₉, X₂₀, X₂₁, X₂₂, X₂₃, X₂₄, and X₂₅ are no amino acid such that the peptide comprises the sequence:

(SEQ ID NO: 8)
GSDGSVGPVGPA

In some aspects, X₁, X₂, X₃, X₄, X₅, X₆, X₇, X₈, and X₉ are no amino acid, X₁₀ is Q, X₁₁ is P, X₁₂ is S, X₁₃ is I, X₁₄ is G, X₁₅ is P, X₁₆ is Q, X₁₇, X₁₈, X₁₉, X₂₀, X₂₁, X₂₂, X₂₃, X₂₄, and X₂₅ are no amino acid such that the peptide comprises the sequence:

(SEQ ID NO: 9)
GPQGPPGSIGPQ

In some aspects, the hydroxyproline-free peptide can include a C-terminus modification selected from: PEG amidification, amidation, glycosyl phosphatidylinositol (GPI), detyrosination, N-alkyl amidification, or esterification with a C₄-C₂₂ fatty acid. In some aspects, the peptide can include a N-terminus modification selected from: fatty acid amidification, urea modification, acetylation, carbamylation, formylation, glycation, methylation, myristoylation, and sulfonamidification.

In some aspects, the hydroxyproline-free peptide can comprise S, T, or Y, and one or more of the S, T, or Y amino acids in the peptide are phosphorylated. In some aspects, the hydroxyproline-free peptide can comprise T and one or more of the T amino acids in the peptide are sulfated. In some aspects, the hydroxyproline-free peptide can comprise C and one or more of the C amino acids in the peptide are prenylated or geranylgeranylated.

In some aspects, the hydroxyproline-free peptide can include a cell-penetrating peptide linked at the amino or carboxy terminus to hydroxyproline-free peptide of this disclosure. The cellular internalization transporter linked to the hydroxyproline-free peptide of this disclosure may be any internalization sequence known or newly discovered in the art, or conservative variants thereof. Non-limiting examples of cellular internalization transporters and sequences include Antennapedia sequences, TAT, HIV-Tat, penetratin, Antp-3A (Antp mutant), buforin II, transportan, MAP (model amphipathic peptide), K-FGF, Ku70, Prion, pVEC, Pep-1, SynB 1, Pep-7, HN-1, BGSC (bis-guanidinium-spermidine-cholesterol, and BGTC (bis-guanidinium-tren-cholesterol). Other sequences of exemplary cellular internalization peptides are can include or exclude: ANTP (RQPKIWFPNRRKPWKK) (SEQ ID NO: 13), HIV-TAT (GRKKRRQRPPQ) (SEQ ID NO: 14), Transportan (GWTLNSAGYLLGKINKALAALAKKIL) (SEQ ID NO: 15), Buforin II (TRSSRAGLQFPVGRVHRLLRK) (SEQ ID NO: 16), Tat (RKKRRQRRR) (SEQ ID NO: 17), Penetratin (RQIKIWFQNRRMKWKK) (SEQ ID NO: 18), MAP (KLALKLALKALKAALKLA) (SEQ ID NO: 19), K-FGF (AAVALLPAVLLALLAP) (SEQ ID NO: 20), Ku70 (VPMLKPMLKE) (SEQ ID NO: 21), Prion (MANLGYWLLALFVTMWTDVGLCKKRPKP) (SEQ ID NO: 22), pVEC (LLIILRRRIRKQAHAHSK) (SEQ ID NO: 23), Pep-1 (KETWWETWWTEWSQPKKKRRV) (SEQ ID NO: 24), SynB 1 (RGGRLSYSRRRFSTSTGR) (SEQ ID NO: 25), Pep-7 (SDLWEMMMVSLACQY) (SEQ ID NO: 26), HN-1 (TSPLNIHNGQKL) (SEQ ID NO: 27), pls1 (RVIRVWFQNKRCKDKK) (SEQ ID NO: 28), MGB Peptide P-beta (GALFLGFLGAAGSTMGAWSQPKKKRKV) (SEQ ID NO: 29), MGB Peptide P-alpha (GALFLAFLAAALSLMGLWSQPKKKRRV) (SEQ ID NO: 30).

In some aspects, the hydroxyproline-free peptide can be connected at the C-terminus, the N-terminus, or both ends, to a tag sequence selected from FLAG (DYKDDDDK) (SEQ ID NO: 31), HA (YPYDVPDYA) (SEQ ID NO: 32), HA12CA5 (CYPYDVPDYA) (SEQ ID NO: 33), Myc (EQKLISEEDL) (SEQ ID NO: 34), His (HHHHHH) (SEQ ID NO: 35), V5 (GKPIPNPLLGLDST) (SEQ ID NO: 36), or RGD (RGD); to a cell-penetrating peptide, a cleavable enzyme substrate sequence (e.g., a mammalian or microbial (e.g., bacterial) protease present in a wound infection), a glycopeptide, a protein (e.g., collagen or gelatin), a glycoprotein, a polysaccharide, a glycosaminoglycan, or a nucleic acid.

In some aspects, the hydroxyproline-free peptides can be connected at the C-terminus, the N-terminus, or both ends, by a linker to one of the aforementioned moieties. In some aspects the linker can be an amino acid or a smaller peptide (i.e., dipeptide, tripeptide), an unnatural amino acid or a similar chemical entity (i.e., β-alanine, 4-aminobutyric acid, (2-aminoethoxy) acetic acid, 5-aminovaleric acid, 6-aminohexanoic acid, p-aminobenzyl alcohol), and polyethylene glycol (PEG)-based spacer (PEG-2 to PEG-12), and combinations thereof.

In some aspects there can be more than one linker functionality attached to the peptide, either on both C- and N-terminus or on only one terminus.

In some aspects the linker(s) could have additional function(s), i.e., serve as cleavage sites to allow for designed release of peptides in specific proteolytic environments. Different linkers can have different enzymatic cleavage susceptibility and be composed of different constituents (e.g., different amino acid cleavage sequence, disulfide, pH-sensitive groups such as esters). Inclusion of one or more proteolytic cleavage sites allows for timely release of the peptide(s). The proteolytic cleavage sites in the described scenarios can be of the same or different sequences; thus allowing for sequential or temporal release of the peptide sequence(s). Similarly, the peptides can be attached to a biodegradable scaffold, using either covalent or non-covalent approaches. Covalent approaches could include use of chemical entities serving as linkers which are cleavable through the means of proteolytic activity, or environmental conditions such as change in pH. Non-covalent approaches could include hydrophobic or charge-based interactions. Both of the approaches have their own unique advantages depending on the environment, application manner, and target of the treatment.

Enzymatically degradable linkages suitable for use in particular embodiments of the present invention can include or exclude: an amino acid sequence cleaved by a serine protease such as thrombin, chymotrypsin, trypsin, elastase, kallikrein, or subtilisin. Illustrative examples of thrombin-cleavable amino acid sequences can include or exclude: -Gly-Arg-Gly-Asp-, -Gly-Gly-Arg-, -Gly-Arg-Gly-Asp-Asn-Pro-, -Gly-Arg-Gly-Asp-Ser-, -Gly-Arg-Gly-Asp-Ser-Pro-Lys-, -Gly-Pro-Arg-, -Val-Pro-Arg-, and -Phe-Val-Arg-. Illustrative examples of elastase-cleavable amino acid sequences can include or exclude: -Ala-Ala-, -Ala-Ala-Pro-Val-, -Ala-Ala-Pro-Leu-, -Ala-Ala-Pro-Phe-, -Ala-Ala-Pro-Ala-, and -Ala-Tyr-Leu-Val-.

Enzymatically degradable linkages suitable for use in particular embodiments of the present invention also include amino acid sequences that can be cleaved by a matrix metalloproteinase such as collagenase, stromelysin, and gelatinase. Illustrative examples of matrix metalloproteinase-cleavable amino acid sequences can include or exclude: -Gly-Pro-Y-Gly-Pro-Z′—, -Gly-Pro-Leu-Gly-Pro-Z′—, -Gly-Pro-Ile-Gly-Pro-Z′—, and -Ala-Pro-Gly-Leu-Z′—, where Y′ and Z′ are amino acids. Illustrative examples of collagenase-cleavable amino acid sequences can include or exclude: -Pro-Leu-Gly-Pro-D-Arg-Z′—, -Pro-Leu-Gly-Leu-Leu-Gly-Z′—, -Pro-Gln-Gly-Ile-Ala-Gly-Trp-, -Pro-Leu-Gly-Cys(Me)-His-, -Pro-Leu-Gly-Leu-Tyr-Ala-, -Pro-Leu-Ala-Leu-Trp-Ala-Arg-, and -Pro-Leu-Ala-Tyr-Trp-Ala-Arg-, where Z′ is an amino acid. An illustrative example of a stromelysin-cleavable amino acid sequence is -Pro-Tyr-Ala-Tyr-Tyr-Met-Arg-; and an example of a gelatinase-cleavable amino acid sequence is -Pro-Leu-Gly-Met-Tyr-Ser-Arg-.

Enzymatically degradable linkages suitable for use in particular embodiments of the present invention also include amino acid sequences that can be cleaved by an angiotensin converting enzyme, such as, for example, -Asp-Lys-Pro-, -Gly-Asp-Lys-Pro-, and -Gly-Ser-Asp-Lys-Pro-.

Enzymatically degradable linkages suitable for use in particular embodiments of the present invention also include amino acid sequences that can be degraded by cathepsin B, such as, for example, Val-Cit, Ala-Leu-Ala-Leu, Gly-Phe-Leu-Gly, and Phe-Lys.

In some aspects the linker(s) could have one or more additional functions to serve to increase or decrease the activity of the peptide through change in hydrophilicity or hydrophobicity.

In some aspects, the linker(s) can have one or more additional functions by increasing the molecular size of the peptide and hence affect its pharmacological activity.

In some aspects, the hydroxyproline-free peptides can be connected by a linker to a glycopeptide, protein, glycoprotein, glycosaminoglycan, or another macromolecular entity. Examples include collagen, gelatin, other hydrolyzed collagen peptides and proteins, polysaccharides, glycoproteins, glycosaminoglycans, nucleic acids.

In some aspects, each of the amino acids in the hydroxyproline-free peptides can be independently substituted with an unnatural amino acid selected from: D-amino acids, citrulline, paphtylanine, alpha-methyl amino acid, d-amino acid, homo-amino acid, selenocysteine, pyrrolysine, ornithine, norleucine, norvaline, 2-amino-heptanoic acid, alpha-aminoisobutyric acid, 2-amino-3-cyclopropylpropanoic acid, cyclohexyl-alanine, alpha-methyl-valine, homo-cysteine, penicillamine, statine, 3-aminobenzoic acid, homophenylalanine, 4-fluorophenylalanine, 5-fluoro-tryptophan, 3-pyridyl-alanine, 3-nitrotyrosine, nitroarginine, pyroglutamic acid, tert-leucine, p-(propargyloxy)phenylalanine, p-methoxyphenylalanine, dansylalanine, DMNB-serine, O-methyl-L-tyrosine, an L-3-(2-naphthyl) alanine, an O-4-allyl-L-tyrosine, an O-propargyl-L-tyrosine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, 3-nitro-L-tyrosine, 4-nitro-L-phenylalanine, 3-amino-L-tyrosine, p-carboxymethyl-Lphenylalanine, biphenyl-alanine, bipyridyl-alanine, 1,5-dansyl-alanine, o-nitrobenzyl-serine, p-cyano-L-phenylalanine, m-cyano-L-phenylalanine, p-ethylthiocarbonyl-L-phenylalanine, p-isopropylthiocarbonyl-L-phenylalanine, 7-amino-coumarine-alanine, and 7-hydroxy-coumarin alanine.

In some aspects, the hydroxyproline-free peptide can be cyclized. The cyclized peptide can be cyclized through the backbone via an amide bond, by cysteine-cysteine crosslinks, by thioether cyclization, by lactam formation, click chemistry with Cu(I)-catalyzed azide-alkynes and strain-promoted azide-alkynes, ligation between tetrazine and alkene, bi-functional PEG linkers, thiol-ene chemistry, native chemical ligation or combinations thereof. The peptide can be head-to-tail, head-to-side chain, tail-to-side chain, or side-chain-to-side chain cyclized with a variety of methodologies forming rigid or flexible bridge. In some instances, there can be more than one cyclization site. In some aspects, the side chains of amino acids in the sequence can be covalently modified, e.g., to facilitate cyclization.

In some aspects, the hydroxyproline-free peptide can be covalently or non-covalently anchored to a biodegradable scaffold. In some aspects, the biodegradable scaffold can be a proteolytic site. The proteolytic site can be sequence-sensitive. The sequence sensitive proteolytic site can be sensitive to a protease present in a microbe or mammal. In some aspects, the microbial protease can be a microbial protease. In some aspects, the microbial protease can be a protease typically present at an infected wound. In some aspects, the microbe can be a bacteria or virus.

In some aspects, the hydroxyproline-free peptide can consist of a peptide of any of SEQ ID Nos. 1-9. In some aspects, the peptide consists of a peptide of any of SEQ ID Nos. 10-11.

In some aspects, this disclosure provides a composition comprising one or more hydroxyproline-free peptides comprising the sequence:

(SEQ ID NO: 12)
X₁X₂X₃X₄X₅X₆X₇X₈X₉GPX₁₀GPX₁₁GX₁₂X₁₃X₁₄X₁₅X₁₆X₁₇X₁₈X₁₉X₂₀X₂₁X₂₂
X₂₃X₂₄X₂₅

wherein X₁, X₂, X₃, X₄, X₅, X₆, X₇, X₈, X₉, X₁₂, X₁₃, X₁₄, X₁₅, X₁₆, X₁₇, X₁₈, X₁₉, X₂₀, X₂₁, X₂₂, X₂₃, X₂₄, and X₂₅ are independently selected from no amino acid, direct bond, G, K, I, S, D, R, E, T, Q, A, V, and P;
wherein X₁₀ is selected from I, R, Q, P, and A;
wherein X₁₁ is selected from V, S, P, R, A, and I;
wherein the peptide is from 9 to 50 amino acids in length;
wherein the peptide does not include hydroxyproline, and
a pharmaceutically acceptable carrier.

In some aspects, the composition can further comprise a mammalian cell. The cells can be selected from autologous or allogeneic human stem cells.

In some aspects, the composition can further comprise a biologically active agent selected from: micronized tissue, morselized tissue, minced tissue, granulated crosslinked bovine tendon collagen, hydrolyzed collagen, amniotic fluid, Wharton's Jelly, or an antimicrobial agent. The biologically active agent can be hydrolyzed collagen and a hydroxyproline-free peptide described herein can be enriched relative to its natural abundance in hydrolyzed collagen.

In some aspects, this disclosure provides a scaffold comprising a peptide as described herein and a structural support agent selected from: collagen, gelatin, glycosaminoglycans, glycoproteins, polysaccharides, polyvinylpyrrolidone, poly(lactic-co-glycolic) acid, polyvinyl alcohol, poly(lactide-co-caprolactone), polycaprolactone, polyhydroxybutyrate, polylactic acid, polyglycolic acid and combinations thereof; silicones, polyolefins, acrylic resins, polyurethanes and combinations thereof. The scaffold can be formed into fibers by a method selected from electrospinning, centrifugal spinning (force spinning), melt blowing, phase separation, self-assembly, template synthesis, drawing, extrusion, interfacial polymerization, and melt-spinning.

In some aspects, the compositions comprising hydroxyproline-free peptides described herein can be utilized in their powder state and can be placed into or on a body defect, wound, burn, or in, on, or surrounding a tissue substitute, wherein the powder mixture can be hydrated by endogenous or exogenous sources.

In some aspects, the compositions of this disclosure can comprise a hydroxyproline-free peptide as described herein and an antimicrobial agent. The antimicrobial agent can be poly(hexamethylene biguanide), copper, or a salt of any of the foregoing.

In some aspects, this disclosure provides a hydroxyproline-free peptide as described herein for use in treating compromised tissue in a subject. In some aspects, this disclosure provides a method of treating compromised tissue, comprising contacting injured or compromised tissue with a treatment composition described herein. The treatment composition can further comprise biologic components such as cells, minced tissue, reactive proteins, polysaccharides, and biologic fluids. In some aspects, the contacting step can comprise applying the treatment composition within tissue, or topical application of the treatment composition. In some aspects, the treatment composition can comprise a liquid carrier. In some aspects, the compromised tissue is at least one of a cut, a wound, a lesion, a fracture, a tear, a break, a rash, a fistula, a burn, a void, a surgical site, an ulcer, or a medical implant site.

In some aspects, this disclosure provides a method of modulating the migration of a fibroblast, keratinocyte, microvascular endothelial cell, macrophage, or pericyte, the method comprising contacting the fibroblast, keratinocyte, or endothelial cell, respectively, with a peptide described herein.

In some aspects, this disclosure provides a use of a hydroxyproline-free peptide as described herein in the manufacture of a medicament for therapeutically treating compromised tissue in a subject.

The compositions of this disclosure can be formulated as skin replacement utilizing tissue engineered skin substitutes and spray-on cells to provide skin repair for compromised tissue which can include or exclude: difficult-to-heal wounds, such as chronic wounds due to diabetes, third-degree burns, and trauma wounds.

In some embodiments, the composition can be coated, injected, sprayed, painted, or implanted in or on tissues, organs, wound void spaces, tissue substitutes, bandages, and medical devices.

### BRIEF DESCRIPTION OF THE DRAWINGS

FIG. 1 shows fibroblast migration in response to treatment with peptide 4.

FIG. 2 shows wound healing results in response to treatment with Peptide 1 and Peptide 2.

FIG. 3 shows wound healing results in response to treatment with Peptide 3 and Peptide 5.

FIG. 4 shows wound healing results in response to treatment with Peptide 6 and Peptide 11.

FIG. 5 shows wound healing results in response to treatment with Peptide 7 and Peptide 10.

FIG. 6 shows wound healing results in response to treatment with Peptide 8 and Peptide 9.

### DETAILED DESCRIPTION

### Definitions

As used herein, “subject” refers to any animal, including humans, domestic and farm animals, and zoo, sports, or pet animals, such as dogs, horses, cats, sheep, pigs, cows, fish, bird, rats, mice, rabbits, and reptiles. The preferred animal herein is a human, including adults, children, and the elderly.

As used herein, “aqueous media” refers to a spectrum of water-based solutions including, but not limited to, homogeneous solutions in water with solubilized components, cell media solutions, buffer solutions, isotonic solutions, salt solutions, emulsified solutions, surfactant solutions, amniotic fluids, Wharton's jelly, serum, blood, plasma, hydrophilic polymers, and viscous or gelled homogeneous or emulsified solutions in water.

As used herein, “biologically active agents” has its standard meaning and includes chemical or biological substances or formulations that beneficially affect human or animal health and well-being or is intended for use in the cure, mitigation, treatment, prevention, or diagnosis of infection or disease, or is destructive to or inhibits the growth of microorganisms.

As used herein, “antimicrobial agent” has its standard meaning and includes a substance that kills microorganisms or inhibits their growth or replication, while an “anti-infective agent” is defined as a substance that counteracts infection by killing infectious agents, such as microorganisms, or preventing them from spreading. Often, the two terms are used interchangeably.

As used herein, “antibiotic” has its standard meaning and includes those substances that were originally produced by a microorganism or synthesized with active properties that can kill or prevent the growth of another microorganism. The term antibiotic is commonly used to refer to almost any prescribed drug that attempts to eliminate infection.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups.

Specific examples of amino acid analogs and mimetics can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983), the entire volume of which is incorporated herein by reference. Other examples include peralkylated amino acids, particularly permethylated amino acids. See, for example, Combinatorial Chemistry, Eds. Wilson and Czarnik, Ch. 11, p. 235, John Wiley & Sons Inc., New York, N.Y. (1997), the entire book of which is incorporated herein by reference. Yet other examples include amino acids whose amide portion (and, therefore, the amide backbone of the resulting peptide) has been replaced, for example, by a sugar ring, steroid, benzodiazepine or carbo cycle. See, for instance, Burger's Medicinal Chemistry and Drug Discovery, Ed. Manfred E. Wolff, Ch. 15, pp. 619-620, John Wiley & Sons Inc., New York, N.Y. (1995), the entire book of which is incorporated herein by reference. Methods for synthesizing peptides, polypeptides, peptidomimetics and proteins are well known in the art (see, for example, U.S. Pat. No. 5,420,109; M. Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984), each of which is incorporated herein by reference).

As used herein, “excipient” has its standard meaning and includes inert substances that form a vehicle, such as a liquid, fluid, or gel, that solubilizes or disperses a hydrolyzed collagen composition, which may include other added ingredients.

As used herein, “hydrophilic” has its standard meaning and includes compounds and materials that have an affinity to water and can be ionic or neutral or have polar groups in their structure that attract water. For example, hydrophilic compounds can be miscible, swellable, adsorbable, or soluble in water, with a stationary contact angle with water of ≤90° in water at room temperature.

As used herein, “hydrophobic” refers to repelling water, being insoluble or relatively insoluble in water, and lacking an affinity for water with a stationary contact angle with water of ≥90° in water at room temperature. Hydrophobic compounds with hydrophilic substituents, such as vicinal dials, may form emulsions in water, with or without added surfactant, with the hydrophilic substituent at the water interface and the hydrophobic portion of the compound in the interior of the emulsion.

As used herein, the term “homology” refers to the percentage number of amino acids that are identical or constitute conservative substitutions. Homology may be determined using common sequence comparison programs or manually.

As used herein, the term “isolated” refers to material that is substantially or essentially free from components that normally accompany it in its native state. An “isolated peptide” or an “isolated polypeptide” as used herein, refers to its meaning as understood in the art and includes the in vitro isolation and/or purification of a peptide or polypeptide molecule from its natural cellular environment, and from association with other components of the cell, i.e., it is not significantly associated with in vivo substances.

As used herein, the term “modulating” includes “increasing,” “stimulating,” “decreasing,” or “reducing,” typically in a statistically significant or a physiologically significant amount as compared to a control. Accordingly, a “modulator” may be an agonist, an antagonist, or any mixture thereof depending upon the conditions used. An “increased” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (the absence of an agent or compound) or a control composition. A “decreased” or reduced amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between.

As used herein, the term “obtained from” is meant that a sample such as, for example, a hydrolyzed peptide extract or polypeptide extract which is isolated from, or derived from, a particular source of the subject. For example, the extract can be obtained from a tissue or a biological fluid isolated directly from the subject. “Derived” or “obtained from” can also refer to the source of a peptide or polypeptide sequence. For instance, a hydroxyproline-free peptide of the present invention may be “derived” from the sequence information of a peptide proteolytic fragment, or a portion thereof, whether naturally-occurring or artificially generated, and may thus comprise, consist essentially of, or consist of that sequence

As used herein, the term “protein” refers to any polymer of two or more individual amino acids (whether or not naturally occurring) linked via amide bonds, as occur when the carboxyl carbon atom of the carboxylic acid group bonded to the alpha-carbon of one amino acid (or amino acid residue) becomes covalently bound to the amino nitrogen atom of the amino group bonded to the alpha-carbon of an adjacent amino acid, and have a molar mass of 12,000 Da or higher. These amide bond linkages, and the atoms comprising them (i.e., alpha-carbon atoms, carboxyl carbon atoms (and their substituent oxygen atoms), and amino nitrogen atoms (and their substituent hydrogen atoms)) form the “polypeptide backbone” of the protein. Similarly, protein fragments, analogs, derivatives, and variants may be referred to herein as “proteins,” and shall be deemed to be a “protein” unless otherwise indicated.

Proteins include protein-based macromolecules and includes extracellular matrices, glycoproteins, structural proteins, fibrous proteins, enzymes, proteoglycans, natural polypeptides, synthetic polypeptides, globular proteins, membrane proteins, plasma proteins, peptides, oligopeptides, antimicrobial peptides, peptide hormones, chaperones, metalloproteins, hemoproteins, coagulation proteins, immune system proteins, ion channel proteins, cell adhesion proteins, neuropeptides, nucleoproteins, scleroproteins, chromoproteins, conjugated proteins, protein-protein complexes, protein-polysaccharide complexes, protein-lipid complexes, protein-enzyme complexes, protein-polymer complexes, motor proteins, mucoproteins, phosphoproteins, contractile proteins, transport proteins, signaling proteins, regulatory proteins, growth factors proteins, sensory proteins, defense proteins, storage proteins, receptor proteins, antibodies, recombinant proteins, fibrinogen, fibrin, thrombin, collagen, elastin, albumin, gelatin, keratin, laminin, and combinations thereof. Hydrolyzed collagens are sufficiently degraded that they are not considered proteins.

As used herein, “peptides” are short chains of two or more amino acids linked by peptide bonds and have a molar mass of 12,000 Da or lower. Peptide chains with twenty or fewer amino acids may also be referred to as oligopeptides. Peptides include hydrolyzed collagen, aeruginosa, cyanopeptolins, microcystins, microviridins, microginins, anabaenopeptins, cyclamides, teprotide, glutathione, and combinations thereof. In addition, as used herein, the term “peptide” is understood to include the terms “polypeptide” (which, at times, may be used interchangeably herein).

As used herein, the term “peptide analogs” refer to the compounds with properties analogous to those of a peptide. “Peptidomimetics” (also known as “mimetic peptides”), which include peptide-based compounds, also include such non-peptide-based compounds such as peptide analogs. Peptidomimetics that are structurally similar to therapeutically useful peptides may be used to produce an equivalent or enhanced therapeutic or prophylactic effect for treating compromised tissue. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. The mimetic can be either entirely composed of natural amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity.

Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid. Partial modification of such an agent may be by way of addition, deletion or substitution of amino acid residues. A substitution may for example be a conserved substitution. Hence a partially modified molecule may be a homologue of the molecule from which it was derived. It may have at least about 40%, for example about 50, 60, 70, 80, 90 or 95%, homology with the molecule from which it is derived.

As used herein, “the hydroxyproline-free peptide hydrolyzed collagen compositions” refer to compositions containing both hydroxyproline-free peptides or peptidomimetics thereof as described herein.

As used herein, “biologically active agents” has its standard meaning and includes chemical or biological substances or formulations that beneficially affect human or animal health and well-being or is intended for use in the cure, mitigation, treatment, prevention, or diagnosis of infection or disease, or is destructive to or inhibits the growth of microorganisms.

As used herein, “antimicrobial agent” has its standard meaning and includes a substance that kills microorganisms or inhibits their growth or replication, while an “anti-infective agent” is defined as a substance that counteracts infection by killing infectious agents, such as microorganisms, or preventing them from spreading. Often, the two terms are used interchangeably.

As used herein, “antibiotic” has its standard meaning and includes those substances that were originally produced by a microorganism or synthesized with active properties that can kill or prevent the growth of another microorganism. The term antibiotic is commonly used to refer to almost any prescribed drug that attempts to eliminate infection.

As used herein, “excipient” has its standard meaning as understood in the art and includes inert substances that form a vehicle, such as a liquid, fluid, or gel, that solubilizes or disperses a hydrolyzed collagen composition, which may include other added ingredients.

As used herein, “pharmaceutically acceptable salts” has its standard meaning as understood in the art and includes salts which are biologically tolerated when administered to a subject. Salts can include cationic, anionic, or zwitterionic salts. Cationic salts include sodium, potassium, ammonium, quaternary ammonium, calcium, magnesium, manganese, arginine, benthazine, chloroprocaine, choline, diethanolamine, ethanolamine, ethylenediamine, lysine, histidine, medlumine, zinc, and lithium salts. Anionic salts include iodide, bromide, chloride, sulfate, sulfite, carbonate, bicarbonate, tosylate, acetate, aspartate, benzenesulfonate, benzoate, besylate, bitartrate, camsylate, citrate, lactobionate, malate, mandelate, mesylate, maleate, mucate, methylsulfate, nitrate, napsylate, octanoate, oleate, pamoate, decanoate, edetate, esylate, fumarate, gluceptate, gluconate, glutamate, glycolate, hexanoate, lactate, salicylate, stearate, succinate, and tartrate.

As used herein, “soft tissue” has its standard meaning and includes biological tissue that connects, supports, or surrounds other structures and organs of the body, but does not include bone. Examples of soft tissue include tendons, ligaments, fascia, skin, fibrous tissues, fat, synovial membranes, muscles, nerves and blood vessels.

As used herein, “matrix” includes for example, matrices such as collagen, acellular matrix, crosslinked biological scaffold molecules, tissue based bioengineered structural framework, biomanufactured bioprosthesis, and other implanted structures such as for example, vascular grafts suitable for cell infiltration and proliferation useful in the promotion of wound healing. Additional suitable biomatrix material may include chemically modified collagenous tissue to reduce antigenicity and immunogenicity. Other suitable examples include collagen sheets for wound dressings, antigen-free or antigen reduced acellular matrix (Wilson et al., Trans Am Soc Artif Intern 1990; 36:340-343) or other biomatrix which have been engineered to reduce the antigenic response to the xenograft material. Other matrix useful in promotion of wound healing may include for example, processed bovine pericardium proteins comprising insoluble collagen and elastin (Courtman et al., J Biomed Mater Res 1994; 28:655-666) and other acellular tissue which may be useful for providing a natural microenvironment for host cell migration to accelerate tissue regeneration (Malone et al., J Vasc Surg 1984; 1:181-91). In certain embodiments, the matrix material may be supplemented with agents useful for wound healing such as growth factors or other wound healing promoting agents for site specific release, therapeutic agents, and/or gap junction modifying agents.

As used herein, the term a “therapeutically effective amount” in reference to the peptides or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve treating and/or repairing comprised tissue (e.g., the promotion of wound healing).

As used herein, the term “treating” refers to both therapeutic treatment and prophylactic or preventative measures. Those in need of treatment include those already with a compromised tissue (e.g., wound or other related disorder) as well as those prone to having a wound or related disorder or diagnosed with the disorder or those in which the disorder is to be prevented.

By “wound” is meant an injury to any tissue, including for example, acute, subacute, delayed or difficult to heal wounds, and chronic wounds. Examples of wounds may include both open and closed wounds. Wounds include, for example, burns, incisions, excisions, lacerations, abrasions, puncture or penetrating wounds, surgical wounds, contusions, hematomas, crushing injuries, and ulcers. Ulcers can be venous ulcers, arterial ulcers, pressure ulcers, vasculitic ulcers, and diabetic ulcers.

In addition to the definition previously provided, the term “wound” may also include injuries to the skin and subcutaneous tissue initiated in different ways (e.g., pressure sores from extended bed rest and wounds induced by trauma) and with varying characteristics.

### Hydrolyzed Collagen Peptides

Hydrolyzed collagen may be derived from acid, alkaline, or enzymatic hydrolysis of collagen (molecular weight of about 300 kDa) to produce hydrolyzed collagen (molecular weight of generally less than 12 kDa). The source of collagen can vary from human, bovine, porcine, piscine, ovine, and avian but can include other sources and can be a mixture of collagen sources. In some embodiments, the hydrolyzed collagen is present at a concentration ranging from 30 wt % to 100 wt %, based on percent solids of the hydroxyproline-free peptide hydrolyzed collagen composition. In some embodiments, the hydrolyzed collagen is present at a concentration ranging from 50 wt % to 99 wt %, or from 60 wt % and 98 wt %.

Surprisingly, and unlike their full-length parental sequences that are known only for their structural function, it has been found that hydrolyzed collagen fragments possess biological activities important for therapeutic applications in treating impaired tissue (e.g., a wound). Embodiments of the present invention therefore include peptides comprising the hydrolyzed hydroxyproline-free peptides of this disclosure, including those having the sequence of SEQ ID Nos; 1-11, in addition to biologically active variants and fragments thereof. In certain embodiments, the peptides may arise through endogenous proteolysis, in vitro proteolysis, splice variation, or in silico prediction, among other mechanisms.

Amino acids typically found in hydrolyzed collagen include alanine, arginine, aspartate, glutamate, glycine, histidine, isoleucine, leucine, lysine, methionine, phenylalanine, proline, hydroxyproline, serine, threonine, tyrosine and valine-plus the presence of glutamic acid and aspartic acid. Glycine, proline and hydroxyproline are concentration predominant in hydrolyzed collagen peptides of this disclosure.

In some embodiments, compositions comprising hydrolyzed collagen peptides of this disclosure can include amino acid dimers and trimers. Pro-Hyp (L-proline, 3-hydroxy-L-proline) is an example of an amino acid dimer that increases cellular activity. Gly-Pro-Hyp (glycine, L-proline, 3-hydroxy-L-proline) and other trimers of the structure Gly-X-Y may also be included. Gly-Pro-Hyp trimers provide local conformational flexibility, which contributes to maintenance of molecular ordering within collagen fibrils) and thus contributes to the structural integrity of cell ligand binding sites. The addition of up to 10 wt % amino acid trimers to the hydroxyproline-free peptide hydrolyzed collagen composition is preferred. Most preferred is the addition of Gly-Pro-Hyp trimer up to 10 wt % based on percent solids of the composition.

In some embodiments, the hydroxyproline-free hydrolyzed collagen peptide composition is blended with collagen or other hydrolyzed collagen peptides. In some embodiments, the hydroxyproline-free hydrolyzed collagen peptide composition comprises at least one of collagen II, collagen IV, and collagen VI. In some embodiments, the hydroxyproline-free hydrolyzed collagen peptide composition comprises each of collagen II, collagen IV, and collagen VI.

### Compositions

In some embodiments, the compositions containing peptide(s) described herein can be aqueous compositions.

In some embodiments, the hydrolyzed collagen component is derived from bovine, porcine, ovine, fowl, marine or mixed sources. Hydrolyzed collagen is generally considered to have a molecular weight range of 75 Da to 12,000 Da and comprises lower molecular weight peptides, oligopeptides, and amino acids. The composition and molecular weight range varies depending on hydrolysis method (acid, alkaline, enzymatic) and processing technique. Hydrolyzed collagen is highly water soluble and is not viscous when dissolved in water until about 60 wt % in water where a water-soluble gel is formed. For the sake of clarity, it is noted that consistent with its standard meaning, hydrolyzed collagen is not a protein.

In some embodiments, the compositions of this disclosure comprise from one or a plurality of hydrolyzed collagen peptides and an excipient. The composition can have from 0.1 to 100 wt. % hydrolyzed collagen peptides. In some embodiments, the weight concentration of the one or a plurality of hydrolyzed collagen peptides in the composition can be 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, 98 wt. % or any concentration between the aforementioned values. When there are two or more hydrolyzed collagen peptides in a composition, the weight concentrations of each of the hydrolyzed collagen peptides can be the same, or different.

In some embodiments, the protein component is or comprises gelatin. The gelatin can be derived from bovine, porcine, ovine, fowl or mixed sources. The molecular weight of the gelatin as provided in Bloom numbers may range, in general, from 150 to 275. However, this range is for demonstration only and may be a wider range (Bloom range from 30 to 325).

In some embodiments, the composition of this disclosure is hydrated with a fluid. In some embodiments, the mobile phase of the fluid is water, isotonic saline, balanced salt solution, buffer solution, Ringer's solution, cell culture media, stem cell media, serum, plasma, amniotic fluid, Wharton's jelly, nutrient broth, antiseptic solutions, or a combination thereof. In some embodiments where the fluid is an aqueous media, the aqueous media can have a pH in the range 4.5 to 8.0, or 5.5 to 7.5. In some embodiments, the fluid is a biological fluid selected from, but not limited to, cell culture media, stem cell media, serum, plasma, amniotic fluid, Wharton's jelly, or nutrient broth.

In some embodiments, the hydroxyproline-free hydrolyzed collagen peptides or peptidomimetics thereof are hydrated with a fluid. In some embodiments, the mobile phase of the fluid is water, isotonic saline, balanced salt solution, buffer solution, Ringer's solution, cell culture media, stem cell media, serum, plasma, amniotic fluid, Wharton's jelly, nutrient broth, antiseptic solutions, or a combination thereof.

In some embodiments, the hydroxyproline-free peptide hydrolyzed collagen composition can include an aqueous solution with amniotic fluid, morselized amniotic tissue, minced tissue, micronized tissue, micronized decellularized tissue, decellularized extracellular matrix derived from stem cells, granulated cross-linked bovine tendon collagen and glycosaminoglycans, cells and stem cells in cell culture medium, synthetic or naturally derived extracellular matrix components, including collagen, glycosaminoglycans, fibrin, laminin, and fibronectin, hydroxyapatite, honey, polysaccharides, biodegradable polymers, including polyglycolides, polylactides, poly(lactide-co-glycolide), polydioxanone, polycaprolactone, poly(trimethylene carbonate), poly(propylene fumarate), polyurethanes, poly(ester amide) s, poly(ortho ester) s, polyanhydrides, poly(amino acid) s, polyphosphazenes, and bacterial polysaccharides and proteins, and combinations thereof, and which can be applied into or on a body.

In some embodiments, the compositions of this disclosure can include an aqueous solution (e.g., carrier fluid) with amniotic fluid, morselized amniotic tissue, exosomes, minced tissue, micronized tissue, decellularized extracellular matrix derived from stem cells, micronized decellularized tissue, plasma, blood, granulated cross-linked bovine tendon collagen and glycosaminoglycans, cells and stem cells in cell culture medium, synthetic or naturally derived extracellular matrix components, including collagen, glycosaminoglycans, fibrin, laminin, and fibronectin, hydroxyapatite, honey, polysaccharides, biodegradable polymers, including polyglycolides, polylactides, poly(lactide-co-glycolide), polydioxanone, polycaprolactone, poly(trimethylene carbonate), poly(propylene fumarate), polyurethanes, poly(ester amide) s, poly(ortho ester) s, polyanhydrides, poly(amino acid) s, polyphosphazenes, and bacterial polyesters, and combinations thereof, and which can be applied to soft tissue, a void or a wound.

In some embodiments, minced tissue can be selected from skin tissue, muscle tissue, vascular tissue, nerve tissue, fat tissue, cartilage tissue, bone tissue, tendon tissue, bladder tissue, intestinal tissue, heart tissue, lung tissue, kidney tissue, liver tissue, pancreatic tissue, and vocal fold tissue. In some embodiments, micronized tissues and micronized decellularized tissues can be selected from skin tissue, muscle tissue, vascular tissue, nerve tissue, fat tissue, cartilage tissue, bone tissue, tendon tissue, bladder tissue, intestinal tissue, heart tissue, lung tissue, kidney tissue, liver tissue, pancreatic tissue, and vocal fold tissue.

In some embodiments, composition of this disclosure includes an antimicrobial agent to hinder development and proliferation of microorganisms. In some embodiments, the addition of an antimicrobial agent helps reduce or eliminate microbial colonies and biofilm formation. Because of the possibility of infection in voids, wounds, and burns, the composition can include a biological agent in an amount sufficient to hinder or eradicate microorganisms. Examples of biological agents can include or exclude, antibiotics, antiseptics, anti-infective agents, antimicrobial agents, antibacterial agents, antifungal agents, antiviral agents, antiprotozoal agents, sporicidal agents, and antiparasitic agents. In some embodiments, the biological agent is biodegradable, non-cytotoxic to human and animal cells, or both biodegradable and non-cytotoxic.

In some embodiments, the composition of this disclosure includes biocidal agents. In some embodiments, the biocidal agents can include or exclude, biguanides, such as poly(hexamethylene biguanide) (PHMB) and its salts, a low molecular weight synthetic cationic biguanide polymer, chlorhexidine and its salts, such as chlorhexidine digluconate, and alexidine and its salts, such as alexidine dihydrochloride, where the latter two are bis(biguanides), benzalkonium chloride, benzethonium chloride, cetyltrimethylammonium bromide, glycerol mono-laurate, capryl glycol, gentamicin sulfate, iodine, povidone iodine, starch-iodine, neomycin sulfate, polymyxin B, bacitracin, tetracyclines, clindamycin, gentamicin, nitrofurazone, mafenide acetate, copper and its salts, silver nanoparticles, silver sulfadiazine, silver nitrate, terbinafine hydrochloride, miconazole nitrate, ketoconazole, clotrimazole, itraconazole, metronidazole, antimicrobial peptides, polyquaternium-1, polyquaternium-6, polyquaternium-10, salts thereof, and combinations thereof.

In some embodiments, the antimicrobial biguanide is poly(hexamethylene biguanide) hydrochloride (PHMB). PHMB can be used because of its high biocidal activity against microorganisms, combined with its biodegradation and low cytotoxicity. PHMB is primarily active against Gram-negative and Gram-positive bacteria, fungi, and viruses. In contrast to antibiotics, which are considered regulated pharmaceutical drugs and to which bacterial resistance can occur, such resistance does not occur with PHMB. As used herein, an “antimicrobial agent” is a substance that kills microorganisms or inhibits their growth or replication, while an “anti-infective agent” is a substance that counteracts infection by killing infectious agents, such as microorganisms, or preventing them from spreading. Often, the two terms are used interchangeably. As used herein, “PHMB” is considered an antimicrobial agent.

In some embodiments, the compositions of this disclosure described herein can include biocidal PHMB at a concentration ranging from 0.0001 wt % (1 ppm) to 1 wt % (10,000 ppm), or ranging from 0.01 wt % (100 ppm) to 0.75 wt % (7,500 ppm), or ranging from 0.05 wt % (500 ppm) to 0.5 wt % (5,000 ppm), or ranging from 0.1 wt % (1,000 ppm) to 0.25 wt % (2,500 ppm), based on the total weight of the composition. In some embodiments, dry hydrolyzed collagen compositions described herein can include biocidal PHMB at a concentration ranging from 0.002 wt % (20 ppm) to 25.0 wt % (250,000 ppm), or ranging from 0.20 wt % (2,000 ppm) to 15.0 wt % (150,000 ppm), or ranging from 1.0 wt % (10,000 ppm) to 10.0 wt % (100,000 ppm), or ranging from 2.0 wt % (20,000 ppm) to 4.0 wt % (40,000 ppm), based on the total weight of the composition.

In some embodiments, bis(biguanide) s, such as alexidine and its salts and chlorhexidine and its salts, can be added to the antimicrobial hydrolyzed collagen compositions in concentrations from 0.001 wt % (10 ppm) to 4.0 wt % (40,000 ppm).

In some embodiments, the compositions of this disclosure can include surfactant-type antimicrobial agents, such as benzethonium chloride or benzalkonium chloride, in concentrations from 0.001 wt % (10 ppm) to 2.0 wt % (20,000 ppm).

In some embodiments, the composition of this disclosure can include lipophilic-type antimicrobial agents, such as glycerol monolaurate or capryl glycol, in concentrations from 0.1 wt % (1,000 ppm) to 2.0 wt % (20,000 ppm).

In some embodiments, the composition of this disclosure can include antimicrobial agents with reactive functional groups, such as amino, imino, imidazoyl, sulfhydryl, hydroxyl, phenolic, indolyl, guanidium, guanidinium, and carboxyl groups. These antimicrobial agents with reactive functional groups can be covalently attached to the peptide sequence.

In some embodiments, the composition can include an aqueous solution (e.g., carrier fluid) with amniotic fluid, morselized amniotic tissue, minced tissue, micronized tissue, micronized decellularized tissue, plasma, blood, granulated cross-linked bovine tendon collagen and glycosaminoglycans, cells and stem cells in cell culture medium, synthetic or naturally derived extracellular matrix components, including collagen, glycosaminoglycans, fibrin, laminin, and fibronectin, hydroxyapatite, honey, polysaccharides, biodegradable polymers, including polyglycolides, polylactides, poly(lactide-co-glycolide), polydioxanone, polycaprolactone, poly(trimethylene carbonate), poly(propylene fumarate), polyurethanes, poly(ester amide) s, poly(ortho ester) s, polyanhydrides, poly(amino acid) s, polyphosphazenes, and bacterial polyesters, and combinations thereof, and which can be injected into compromised tissue (which can include or exclude soft tissue, a void or a wound).

In some embodiments, minced tissue can be selected from skin tissue, muscle tissue, vascular tissue, nerve tissue, fat tissue, cartilage tissue, bone tissue, tendon tissue, bladder tissue, intestinal tissue, heart tissue, lung tissue, kidney tissue, liver tissue, pancreatic tissue, and vocal fold tissue. In some embodiments, micronized tissues and micronized decellularized tissues can be selected from skin tissue, muscle tissue, vascular tissue, nerve tissue, fat tissue, cartilage tissue, bone tissue, tendon tissue, bladder tissue, intestinal tissue, heart tissue, lung tissue, kidney tissue, liver tissue, pancreatic tissue, and vocal fold tissue.

In some embodiments, one or more biologically active agents may be incorporated into the composition of this disclosure to provide a medical benefit to a mammalian host. Examples of biologically active agents that can be incorporated into the composition can include or exclude, cells, stem cells, amniotic tissue, amniotic cells, growth factors, micronized decellularized skin tissue, granulated crosslinked bovine tendon collagen and glycosaminoglycans, antibiotics, antiseptics, anti-infective agents, antimicrobial agents, antibacterial agents, antifungal agents, antiviral agents, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), anti-VEGF RNA aptamer, nucleic acids, DNA, DNA fragments, DNA plasmids, Si-RNA, transfection agents, vitamins, essential oils, liposomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, antibiotic-containing nanoparticles, nitric oxide-containing nanoparticles, nanoshells, nanorods, polymeric micelles, silver salts, zinc salts, quantum dots nanoparticles, polymer-based microparticles, polymer-based microspheres, drug-containing microparticles, drug-containing microspheres, antibiotic-containing microparticles, antibiotic-containing microspheres, antimicrobial microparticles, antimicrobial microspheres, salicylic acid, benzoyl peroxide, 5-fluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, oligopeptides, cyclopeptides, polypeptides, proteins, prodrugs, protease inhibitors, cytokines, hyaluronic acid, chondroitin sulfate, dermatan sulfate, para-sympatholytic agents, chelating agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle. Such biologically active agents could be in either of the (R)-, (R, S)-, or (S)-configuration, or a combination thereof.

In some embodiments, the composition may include cells. Examples of cells useful in the hydrolyzed collagen compositions described herein can include or exclude, fibroblasts, keratinocytes, neurons, glial cells, astrocytes, Schwarm cells, dorsal root ganglia, adipocytes, endothelial cells, epithelial cells, chondrocytes, fibrochondrocytes, myocytes, cardiomyocytes, myoblasts, hepatocytes, tenocytes, intestinal epithelial cells, smooth muscle cells, stromal cells, neutrophils, lymphocytes, bone marrow cells, platelets, and combinations thereof. In some embodiments, the cells are eukaryotic or mammalian. In some embodiments, the cells are of human origin. In some embodiments, the cells may be autologous or allogeneic.

In some embodiments the composition may include adult mammalian stem cells, embryonic stem cells, amniotic stem cells, induced pluripotent stem cells, fetal stem cells, tissue stem cells, adipose-derived stem cells, bone marrow stem cells, human umbilical cord blood stem cells, blood progenitor cells, mesenchymal stem cells, hematopoietic stem cells, epidermal stem cells, endothelial progenitor cells, epithelial stem cells, epiblast stem cells, cardiac stem cells, pancreatic stem cells, neural stem cells, limbal stem cells, perinatal stem cells, satellite cells, side population cells, multipotent stem cells, totipotent stem cells, unipotent stem cells, and combinations thereof. In some embodiments, the stem cells are mammalian. In some embodiments, the stem cells are of human origin. In some embodiments, the stem cells may be autologous or allogeneic.

In some embodiments, the hydroxyproline-free hydrolyzed collagen peptides or peptidomimetics thereof described herein may be used as a scaffold matrix to deliver a therapeutically effective amount of between 10,000 cells to about 1 billion or more cells. In some embodiments, products derived from placental tissue may be incorporated with the composition for placement into a mammalian host. Placental tissues are a source of collagen, elastin, fibronectin, and growth factors, including platelet-derived growth factor (PDGF), vascular endothelial growth factor (VEGF), epidermal growth factor (EGF), fibroblast growth factor (FGF), and transforming growth factor beta (TGF-β), which can support tissue repair and regeneration. In particular, amniotic tissue has anti-adhesive and antimicrobial properties, and such tissue has been shown to support soft tissue repair, reduce inflammation and minimize scar tissue formation, which are significant benefits in the treatment of soft tissue injuries.

Amniotic tissues have been described as immune-privileged in that an immune response in the human body rarely occurs in response to the introduction of amniotic tissue. In some embodiments a morselized, flowable tissue allograft derived from amniotic tissues can be added to the composition for a coating or injection into soft tissue, or placement surrounding a tissue substitute.

In some embodiments, the composition may include growth factors. Examples of useful growth factors can include or exclude, epidermal growth factor (EGF), transforming growth factor beta (TGF-β), fibroblast growth factor (FGF), vascular endothelial growth factor (VEGF), granulocyte macrophage colony stimulating factor (GM-CSF), platelet-derived growth factor (PDGF), connective tissue growth factor (CTGF), insulin-like growth factor (IGF), keratinocyte growth factor (KGF), interleukin (IL) family, stromal cell derived factor (SDF), heparin binding growth factor (HBGF), nerve growth factor (NGF), brain-derived neurotrophic factor (BDNF), growth differentiation factor (GDF), muscle morphogenic factor (MMF), tumor necrosis factor-alpha (TNFα), and bone morphogenetic proteins (BMP).

In some embodiments, the composition of this disclosure can be used as an injectable biological tissue void filler and may also include any other component suitable for augmenting, strengthening, supporting, repairing, rebuilding, healing, occluding or filling biological tissue.

In some embodiments, the liquid carrier can be selected from the group consisting of, but not limited to, deionized water, water, plasma, blood, methanol, etc.

In some embodiments, the compositions of this disclosure can comprise a chelating agent. In some embodiments, the compositions of this disclosure comprises a chelating agent selected from the group consisting of aminocarboxylic acids, citric acid and its salts, ethylenediaminetetraacetic acid (EDTA), nitrilotriacetic acid, nitrilotripropionic acid, diethylenetriaminepentaacetic acid, 2-hydroxyethylethylenediaminetriacetic acid, 1,6-diaminohexamethylenetetraacetic acid, 1,2-diaminocyclohexanetetraacetic acid, O,O′-bis(2-aminoethyl)ethyleneglycoltetraacetic acid, 1,3-diaminopropanetetraacetic acid, N,N-bis(2-hydroxybenzyl)ethylenediamine-N,N-diacetic acid, ethylenediamine-N,N-diacetic acid, ethylenediamine-N,N-dipropionic acid, triethylenetetraaminehexaacetic acid, ethylenediamine-N,N-bis(methylenephosphonic acid), iminodiacetic acid, N,N-bis(2-hydroxyethyl)glycine, 1,3-diamino-2-hydroxypropanetetraacetic acid, 1,2-diaminopropanetetraacetic acid, ethylenediaminetetrakis(methylenephosphonic acid), N-(2-hydroxyethyl)iminodiacetic acid and biphosphonates. In some embodiments, the hydrolyzed collagen-based composition comprises EDTA.

In some embodiments, the compositions of this disclosure comprises 0.01 to 5 wt %, or 0.05 to 3 wt % of the chelating agent based on the percent solids of the composition.

In some embodiments, the composition of this disclosure comprises a biologically active agent selected from cells, stem cells, amniotic tissue, amniotic cells, exosomes, growth factors, micronized decellularized tissue, decellularized extracellular matrix derived from stem cells, granulated crosslinked bovine tendon collagen and glycosaminoglycans, antiprotozoal agents, sporicidal agents, antiparasitic agents, peripheral neuropathy agents, neuropathic agents, chemotactic agents, analgesic agents, anti-inflammatory agents, anti-allergic agents, anti-hypertension agents, mitomycin-type antibiotics, polyene antifungal agents, antiperspirant agents, decongestants, anti-kinetosis agents, central nervous system agents, wound healing agents, anti-VEGF agents, anti-tumor agents, escharotic agents, anti-psoriasis agents, anti-diabetic agents, anti-arthritis agents, anti-itching agents, antipruritic agents, anesthetic agents, anti-malarial agents, dermatological agents, anti-arrhythmic agents, anti-convulsants, antiemetic agents, anti-rheumatoid agents, anti-androgenic agents, anthracyclines, anti-smoking agents, anti-acne agents, anticholinergic agents, anti-aging agents, antihistamines, anti-parasitic agents, hemostatic agents, vasoconstrictors, vasodilators, thrombogenic agents, anti-clotting agents, cardiovascular agents, angina agents, erectile dysfunction agents, sex hormones, growth hormones, isoflavones, integrin binding sequences, biologically active ligands, cell attachment mediators, immunomodulators, tumor necrosis factor alpha, anti-cancer agents, anti-depressant agents, antitussive agents, anti-neoplastic agents, narcotic antagonists, anti-hypercholesterolemia agents, apoptosis-inducing agents, birth control agents, sunless tanning agents, emollients, alpha-hydroxyl acids, manuka honey, topical retinoids, hormones, tumor-specific antibodies, antisense oligonucleotides, small interfering RNA (siRNA), mRNA, anti-VEGF RNA aptamer, nucleic acids, DNA, DNA fragments, DNA plasmids, transfection agents, vitamins, essential oils, liposomes, exosomes, silver nanoparticles, gold nanoparticles, drug-containing nanoparticles, albumin-based nanoparticles, chitosan-containing nanoparticles, polysaccharide-based nanoparticles, dendrimer nanoparticles, phospholipid nanoparticles, iron oxide nanoparticles, bismuth nanoparticles, gadolinium nanoparticles, metallic nanoparticles, ceramic nanoparticles, silica-based nanoparticles, virus-based nanoparticles, virus-like nanoparticles, nitric oxide-containing nanoparticles, nanoshells, nanorods, polymeric micelles, quantum dots nanoparticles, polymer-based microparticles, polymer-based microspheres, drug-containing microparticles, drug-containing microspheres, salicylic acid, benzoyl peroxide, 5-fluorouracil, nicotinic acid, nitroglycerin, clonidine, estradiol, testosterone, nicotine, motion sickness agents, scopolamine, fentanyl, diclofenac, buprenorphine, bupivacaine, ketoprofen, opioids, cannabinoids, enzymes, enzyme inhibitors, proteins, prodrugs, protease inhibitors, hyaluronic acid, chondroitin sulfate, dermatan sulfate, para-sympatholytic agents, hair growth agents, lipids, glycolipids, glycoproteins, endocrine hormones, growth hormones, growth factors, differentiation factors, heat shock proteins, immunological response modifiers, saccharides, polysaccharides, insulin and insulin derivatives, steroids, corticosteroids, and non-steroidal anti-inflammatory drugs or similar materials, in either their salt form or their neutral form, either being inherently hydrophilic or encapsulated within a hydrophilic microparticle or nanoparticle.

In some embodiments, the compositions of this disclosure comprise at least one of cells, stem cells, amniotic tissue, amniotic cells, exosomes, growth factors, micronized decellularized tissue, granulated collagen, gelatin, or glycosaminoglycans. In some embodiments, the cells are animal cells. In some embodiments, the cells can be mammalian cells or non-mammalian cells.

In some embodiments, the compositions of this disclosure comprises at least one additional ingredient selected from glycolipids, glycoproteins, immunological response modifiers, saccharides, and polysaccharides.

In some embodiments, the compositions of this disclosure has a form selected from a powder, a liquid, a gel, a paste, a cream, a suspension, an emulsion, a film, a sheet, a foam, a lotion, a spray, an aerosol, a capsule, or a tablet. In some embodiments, the composition is a powder.

In some embodiments, the hydrolyzed collagen peptide(s) may be incorporated in the compositions using a variety of methods such as blending as a powder or as a solution, using a solvent such as aqueous media. If a solution is used, the solvent can be evaporated or can remain in the final form.

In some embodiments, the peptide powder can be mixed with other dry solid components. The powder can then be applied directly. Alternately, the powder can be dissolved (e.g., within a syringe) in an aqueous solvent, then applied as appropriate.

In some embodiments, the peptide powder can be mixed with other powder components, then dissolved in an aqueous solvent, and then dried and formulated into a combined powder.

In some embodiments, the compositions of this disclosure can be mixed with polar liquids, such as alcohols and water, and applied to or within living tissue; or in, on, or surrounding a device (e.g., an implant device).

In some embodiments, the compositions of this disclosure may be applied using microneedles.

In some embodiments, the compositions of this disclosure can be aqueous compositions. As used herein, “aqueous” compositions can include or exclude, solutions in water or aqueous media with solubilized components, emulsified solutions in water stabilized by surfactants or hydrophilic polymers, as well as, viscous or gelled homogeneous or emulsified solutions.

In some embodiments, the compositions of this disclosure can be utilized in a powder state and can be placed into or on a tissue defect, wound, burn, or in, on, or surrounding a medical device (e.g., an implanted medical device or medical device prior to implantation). In some embodiments, the powder mixture can be hydrated by endogenous or exogenous fluid sources.

In some embodiments where the compositions of this disclosure is an aqueous-based solution, gel, paste, emulsion, or foam, a water-soluble polymer can be added to increase solution viscosity and to prolong residence time on the surface of a tissue, void, or wound, or subcutaneously in a void or wound. In some embodiments, useful water-soluble polymers can include or exclude, poly(ethylene glycol), poly(ethylene oxide), poly(vinyl alcohol) and copolymers, poly(N-vinylpyrrolidone) and copolymers, methylcellulose, hydroxyethylcellulose, hydroxypropylcellulose, hydroxypropylmethylcellulose, carboxymethylcellulose, guar gum, hydroxyethylguar, hydroxypropylguar, gelatin, albumin, hydroxypropylmethylguar, carboxymethylguar, carboxymethylchitosan, locust bean gum, carrageenan, xanthan gum, gellan gum, pullulan, alginate, chondroitin sulfate, dextran, dextran sulfate, Aloe vera gel, scleroglucan, schizophyllan, gum arabic, tamarind gum, poly(methyl vinyl ether), ethylene oxide-propylene oxide-ethylene oxide block copolymers, hyaluronan, chondroitin sulfate, keratan sulfate, dermatan sulfate, heparan sulfate, dextran, carbomer and its salts, poly(acrylic acid) and its salts, poly(methacrylic acid) and its salts, poly(ethylene-co-acrylic acid), poly(vinyl methyl ether), poly(vinylphosphoric acid) salts, poly(vinylsulfonic acid) salts, sodium poly(2-acrylamido-2-methylpropanesulfonate), polyacrylamide(s), poly(N,N′-dimethylacrylamide), poly(N-vinylacetamide), poly(N-vinylformamide), poly(2-hydroxyethyl methacrylate), poly(glyceryl methacrylate), poly(2-ethyl-2-oxazoline), poly(N-isopropylacrylamide) and poly(N-vinylcaprolactam), the latter two hydrated below their Lower Critical Solution Temperatures, polyquaternium-1, polyquaternium-6, poly-quaternium-10, ionene polymers, cationic guar, pyridinium polymers, imidazolium polymers, diallyldimethylammonium polymers, acryloyl-, methacryloyl-, and styryl-trimethylammonium polymers, acrylamido- and methacrylamido-trimethylammonium polymers, and the like, and derivatives and combinations thereof.

In some embodiments, the preparation of compositions containing peptide(s) in the form of viscous solutions, gels, creams, pastes, emulsions, balms, and sprays, can be facilitated by the inclusion of water-soluble polymer viscosity builders in amounts ranging from about 0.01 to about 50.0 wt %, from 0.1 to 45% wt, from 0.5 to 25 wt %, or from 1.0 to 10.0 wt %.

In some embodiments, essential oils can be added to the compositions containing peptide(s) as fragrance or aromatic agents, and/or as antimicrobial agents. Examples of essential oils useful in the compositions described herein can include or exclude, thymol, menthol, sandalwood, camphor, cardamom, cinnamon, jasmine, lavender, geranium, juniper, menthol, pine, lemon, rose, eucalyptus, clove, orange, oregano, mint, linalool, spearmint, peppermint, lemongrass, bergamot, citronella, cypress, nutmeg, spruce, tea tree, wintergreen (methyl salicylate), vanilla, and the like. In some embodiments, the essential oils can be selected from thymol, sandalwood oil, wintergreen oil, eucalyptol, pine oil, and combinations thereof. In some embodiments, essential oils can be present in the compositions in an amount ranging from 0% to 5 wt % based on the total weight of the composition. In some embodiments, essential oils can be present in the composition in an amount of at least 0.1 wt %, or at least 0.25 wt %, or at least 0.5 wt %, based on the weight of the composition.

In some embodiments, chlorophyllin, a water-soluble semi-synthetic derivative of chlorophyll, may also be used to control wound odor and to provide anti-inflammatory properties. In some embodiments, chlorophyllin can be present in an amount ranging from 0% to 5 wt % based on the weight of the composition. In some embodiments, chlorophyllin can be present in an amount of at least 0.1 wt %, or at least 0.25 wt %, or at least 0.5 wt % based on the weight of the composition.

In some embodiments, the compositions of this disclosure can also include wetting agents, buffers, gelling agents or emulsifiers. Other excipients could include various water-based buffers ranging in pH from 5.0-7.5, surfactants, silicones, polyether copolymers, vegetable and plant fats and oils, hydrophilic and hydrophobic alcohols, vitamins, monoglycerides, laurate esters, myristate esters, palmitate esters, and stearate esters.

### Hydrolyzed Collagen Peptides for Treating Impaired Tissue

The wound healing process depends on the migration and proliferation of cells at or near the wound edge, as well as recruitment or formation of new blood vessels at the wound site. Described here are the effects of the bioinductive wound healing peptides which stimulate motility and/or proliferation of some of the key cell types involved in the wound healing and tissue regeneration process.

As used herein, the terms “bioinductive wound healing peptides”, “wound healing peptides” and “peptides” as used in the specification and the appended claims are the sequence ID numbers 1-11 and their derivatives.

Cell types which can be directed or influenced by these peptides include fibroblasts, keratinocytes, microvascular endothelial cells, macrophages, pericytes, to name a few. These peptides can influence a multitude of processes associated with wound healing, such as cell proliferation, cell migration, angiogenesis, as well as expression profiles of (pro- and anti-) inflammatory markers, cytokines and chemokines. Individual peptides can affect a specific cell type and/or process, or multiple cell types and/or processes. By extension, a combination of individual peptides can have an additive or a synergistic effect on cells, tissues, and processes evaluated. Additionally, compositions which include peptide(s) and other bioactive entities can also exhibit additive or a synergistic effect on cells, tissues, and processes evaluated.

In some embodiments, the compositions of this disclosure enhances a natural tissue regeneration process. In some embodiments, the natural tissue regeneration processes that is enhanced is at least one of cell migration, cell proliferation, cell viability, cell differentiation, and angiogenesis. In some embodiments, the cells are fibroblasts. In some embodiments, the cells are keratinocytes. In some embodiments, the cells are endothelial cells. In some embodiments, the cells are macrophages. In some embodiments, the cells are pericytes. As used herein, a natural tissue regeneration process is enhanced if the property is improved compared to an untreated control.

In some embodiments, at least one natural tissue regeneration process is improved by at least 5%, or at least 10%, or at least 15%, or at least 20%, or at least 25% compared to an untreated control.

In some embodiments the compositions containing peptide(s) facilitate repair of injured or compromised tissues. Examples of injured or compromised tissue can include or exclude, surgical incision sites, lesions, fissures, fistulas and diverticula. The tissue can be injured or compromised physiologically or as the result of infection, surgery, cyst, tumor removal, or traumatic injury or remodeling of soft tissue, such as in skin and wound healing, plastic surgery, cosmetic surgery, reconstructive surgery, coating/sealing of skin replacement products, tendon repair, hernia repair, craniofacial surgery, ophthalmic surgery, cervicofacial rhytidectomy, abdominoplasty, breast augmentation, myocardium repair, nerve repair, spinal cord repair, liver tissue regeneration, bladder repair, muscle repair, mastopexy, rheumatology, gynecomastia reduction, body contouring, skin rejuvenation, skin resurfacing, microsurgery, dermato-cosmetics for filling in wrinkles, masking scars or enhancing lips, and the like.

In some embodiments, this disclosure provides for a method of treating compromised tissue. The method includes contacting injured or compromised tissue with a treatment composition comprising a composition as described herein.

In some treatment embodiments, the treatment composition further comprises biologic components such as cells, minced tissue, reactive proteins, polysaccharides, and biologic fluids.

In some treatment embodiments, the contacting step comprises applying the treatment composition within or on tissue. In some embodiments, the tissue is compromised tissue. In some embodiments, the treatment composition is applied subcutaneously. In some embodiments, the treatment is applied via injection. In some embodiments, the treatment is applied using a syringe (without a needle) or tube.

In some treatment embodiments, the contacting step comprises topical application of the treatment composition.

In some treatment embodiments, the treatment composition comprises a hydroxyproline-free hydrolyzed collagen peptides or peptidomimetics thereof in a liquid carrier.

In some treatment embodiments, the compromised tissue is at least one of a cut, a wound, a lesion, a fracture, a tear, a break, a rash, a fistula, a burn, a void, a blunt injury site, a biopsy site, a surgical site, or a medical implant site.

Also disclosed are uses of the hydroxyproline-free hydrolyzed collagen peptides or peptidomimetics thereof described herein for treating a comprised tissue (e.g., wound). Any of the hydroxyproline-free hydrolyzed collagen peptides or peptidomimetics thereof described herein can be used. In particular, the compositions can be used for injection into biological tissue. The injection can be intradermal, subcutaneous, oral, intramuscular, submucosal, intranasal, vaginal, buccal, intrathecal, epidural, intraparenchymal, ocular, subretinal, dental, intra-tumoral, intracardiac, intra-articular, intravenous, intra-cavernous, intraosseous, intraperitoneal, intra-abdominal, intra-fascial, intra-organ, and intravitreal. In some embodiments, the hydrolyzed collagen compositions described herein are in the form of a dry powder, and the use and or method further comprises hydrating said dry powder prior to said injection step.

In some embodiments, this disclosure relates to methods of using hydroxyproline-free hydrolyzed collagen peptides or peptidomimetics thereof described herein to treat subjects suffering from or at risk for various diseases, disorders, and conditions associated with a compromised tissue, including a wound.

Preferred methods include the sequential or simultaneous administration of one or more hydroxyproline-free hydrolyzed collagen peptides or peptidomimetics thereof compositions and one or more agents useful for treating compromised tissue, either or both of which are provided in amounts or doses that are less that those used when the agent or agents are administered alone, i.e., when they are not administered in combination, either physically or in the course of treatment of a compromised tissue. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the administration is sequential. Preferably, the agents are administered sequentially within at least about one-half hour of each other. The agents may also be administered within about one hour of each other, within about one day to about one week of each other, or as otherwise deemed appropriate. Preferably, the hydroxyproline-free hydrolyzed collagen peptides or peptidomimetics thereof are administered first. Preferably, one or more hydroxyproline-free hydrolyzed collagen peptides or peptidomimetics thereof are administered prior to the administration of the one or more agents useful for treating compromised tissue.

In some embodiments, this disclosure relates to transdermal patches, dressings, pads, wraps, matrices and bandages capable of being adhered to or otherwise associated with the skin of a subject, said articles being capable of delivering a therapeutically effective amount of one or more hydroxyproline-free hydrolyzed collagen peptides or peptidomimetics thereof described herein.

In some embodiments, this disclosure relates to an article of manufacture comprising a vessel containing a therapeutically effective amount of one or more hydroxyproline-free hydrolyzed collagen peptides or peptidomimetics thereof for promotion of wound healing and instructions for use, including use for the treatment of a subject.

In some embodiments, this disclosure relates to a formulation comprising hydroxyproline-free hydrolyzed collagen peptides or peptidomimetics thereof of this disclosure in amounts effective to promote wound healing in a subject. This disclosure includes a formulation comprising hydroxyproline-free hydrolyzed collagen peptides or peptidomimetics thereof tide in amounts effective to promote wound healing in a subject. Such formulations include, for example, topical delivery forms and formulations. Preferred formulations include one or more hydroxyproline-free hydrolyzed collagen peptides or peptidomimetics thereof and one or more agents useful for wound healing, either or both of which are provided in amounts or doses that are less than those used when the agent or agents are administered alone, i.e., when they are not administered in combination, either physically or in the course of treatment of a wound. Such lesser amounts of agents administered or provided in combination are typically from about one-twentieth to about one-tenth the amount or amounts when administered alone, and may be about one-eighth the amount, about one-sixth the amount, about one-fifth the amount, about one-fourth the amount, about one-third the amount, and about one-half the amount when administered alone.

In some embodiments, this disclosure relates to the use of therapeutically effective amounts of compositions comprising one or more hydroxyproline-free hydrolyzed collagen peptides or peptidomimetics thereof useful for treating compromised tissue (e.g., wound healing) in the manufacture of a medicament. Such medicaments include, for example, topical delivery forms and formulations. Such medicaments include those for the treatment of a subject as disclosed herein. Such medicaments preferably include the reduced amounts of the one or more hydroxyproline-free hydrolyzed collagen peptides or peptidomimetics thereof, as noted herein.

In some embodiments, this disclosure relates to a method of promoting or improving wound healing in a subject having a wound which comprises sustained administration of hydroxyproline-free hydrolyzed collagen peptides or peptidomimetics thereof, composition or formulation of the present invention and one or more agents useful for wound healing, in an amount effective to increase re-epithelialization rates in the wound area. In one embodiment the method comprises sustained administration of a peptide of SEQ ID Nos: 1-11 or a composition or formulation comprising such. In one embodiment, the composition or compositions are administered in a sustained release formulation. In another embodiment, the composition or compositions are administered for a sustained period of time.

In another aspect, the invention provides for the use of hydroxyproline-free hydrolyzed collagen peptides or peptidomimetics thereof useful for wound healing, in the manufacture of a pharmaceutical product for the treatment of compromised tissue (e.g., promotion of wound healing) in a patient in need thereof.

### Dosage Forms and Formulations and Administration

A therapeutically effective amount of each of the hydroxyproline-free hydrolyzed collagen peptides or peptidomimetics thereof may be administered simultaneously, separately or sequentially and in any order. The agents may be administered separately or as a fixed combination. When not administered as a fixed combination, preferred methods include the sequential administration of one or more hydroxyproline-free hydrolyzed collagen peptides or peptidomimetics thereof useful for wound healing, either or both of which are provided in amounts or doses that are less that those used when the agent or agents are administered alone, i.e., when they are not administered in combination, either physically or in the course of treatment of a wound.

The agents of the invention may be administered to a subject in need of treatment, such as a subject with any of the diseases or conditions mentioned herein. The condition of the subject can thus be improved. The hydroxyproline-free hydrolyzed collagen peptides or peptidomimetics thereof thus be used in the treatment of the subject's body by therapy. They may be used in the manufacture of a medicament to treat any of the conditions mentioned herein.

The hydroxyproline-free hydrolyzed collagen peptides or peptidomimetics thereof may be present in a substantially isolated form. It will be understood that the product may be mixed with carriers or diluents which will not interfere with the intended purpose of the product and still be regarded as substantially isolated. A product of the invention may also be in a substantially purified form, in which case it will generally comprise about 90%, e.g. at least about 95%, at least about 98% or at least about 99% of the hydroxyproline-free hydrolyzed collagen peptides or peptidomimetics thereof or dry mass of the preparation.

Depending on the intended route of administration, the pharmaceutical products, pharmaceutical compositions, combined preparations and medicaments of the invention may, for example, take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose (CMC)-based formulations, and hydroxypropylmethylcellulose (HPMC)-based formulations. Other useful formulations include slow or delayed release preparations.

Gels or jellies may be produced using a suitable gelling agent including, but not limited to, gelatin, tragacanth, or a cellulose derivative and may include glycerol as a humectant, emollient, and preservative. Ointments are semi-solid preparations that consist of the active ingredient incorporated into a fatty, waxy, or synthetic base. Examples of suitable creams can include or exclude, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base can include or exclude, propylene glycol, emulsifying wax, cetyl alcohol, and glyceryl stearate. Potential preservatives include phenyl ethanol and PHMB.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing or pH buffering agents may also be present.

The effective dose for a given subject or condition can be determined by routine experimentation or other methods known in the art or later developed. For example, in order to formulate a range of dosage values, 2D and 3D cell culture assays and animal studies can be used. The dosage of such compounds preferably lies within the dose that is therapeutically effective for at least 50% of the population, and that exhibits little or no toxicity at this level.

The effective dosage of each of the hydroxyproline-free hydrolyzed collagen peptides or peptidomimetics thereof employed in the methods and compositions of the invention may vary depending on a number of factors including the particular hydroxyproline-free hydrolyzed collagen peptides or peptidomimetics thereof employed, the combinational partner, the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient and the particular hydroxyproline-free hydrolyzed collagen peptides or peptidomimetics thereof that are being administered.

### EXAMPLES

### Example 1

Representative hydroxyproline-free peptides of this disclosure having the sequences of SEQ ID Nos: 1-11 as described herein were isolated from bovine collagens subjected to a series of treatment procedures, including enzymatic hydrolysis and were also independently synthesized de novo. As such, these peptides can be isolated from digested collagen mixture, digested tissues, or prepared using several available methodologies, including conventional peptide synthesis and recombinant methodologies. In some cases, a combination of methodologies listed can be employed.

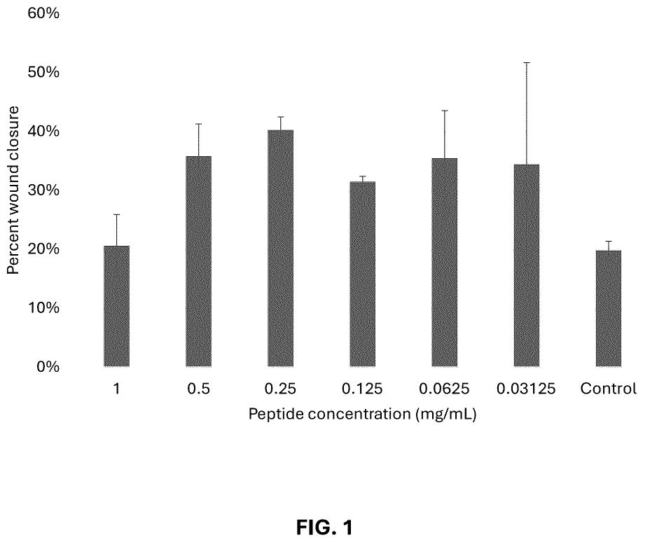

As demonstrated in FIG. 1, in an in vitro assay, peptide 4 (GPIGPVGAR-NH2) (SEQ ID NO: 4) exhibits dose-dependent migration of primary human dermal fibroblasts with the wound closure up to 40% at the designated time point (6 h) compared to approximately 20% wound closure for the control group (medium only). This constitutes up to a 100% improvement in fibroblast migration with the inclusion of peptide 4.

The methods, kits, and compositions described herein can include at least one of the peptides from the sequence ID numbers 1-11, or can include a mixture of those peptides, or one or more of the listed peptides can be included in the final composition which includes other components, be it excipients, carriers, or entities with bioactive properties.

In some embodiments, protein fragments such as endogenous proteolytic hydroxyproline-free peptides, however generated, can be identified by techniques such as mass-spectrometry, or equivalent techniques. Once an in vitro or endogenously identified protein fragment has been generated or identified, it can be mapped or sequenced, and, for example, cloned into an expression vector for recombinant production, or produced synthetically.

TABLE 1
Sequences of Hydroxyproline-free peptides from
hydrolyzed collagen.

| Se- | | | |
|---|---|---|---|
| quence | | | |
| ID No | Length | Organism | Sequence |
| 1 | 21 | Bos taurus | GKSGDRGETGPAGPAGPIGPV |
| 2 | 24 | Bos taurus | GKSGDRGETGPAGPAGPIGPV |
| | | | GAR |
| 3 | 12 | Bos taurus | GPAGPIGPVGAR |
| 4 | 9 | Bos taurus | GPIGPVGAR |
| 5 | 18 | Bos taurus | GEGGPQGPRGSEGPQGVR |
| 6 | 27 | Bos taurus | GPRGPAGPSGPAGKDGRIGQP |
| | | | GAVGPA |
| 7 | 12 | Bos taurus | GAVGPRGPSGPQ |
| 8 | 12 | Bos taurus | GSDGSVGPVGPA |
| 9 | 12 | Bos taurus | GPQGPPGSIGPQ |
| 10 | 9 | Bos taurus | GPPGSPGPA |
| 11 | 19 | Bos taurus | PGTSGPPGLAGPPGSPGPQ |

Materials: Peptides with sequence IDs 1-11 with a C-terminal modification (amidation) were synthesized by GenScript (Piscataway, NJ) and used without further modifications. The identity and purity of the peptides were confirmed using mass spectrometry and reversed phase high pressure liquid chromatography (RP-HPLC). Primary human dermal fibroblasts (HDFa Cat. No. 201-012, Lot No. 80405992 and 8092232) were purchased from ATCC (Manassas, VA) and cultured according to the manufacturer's recommendations. Media: ATCC Fibroblast, Cat: 201-030 Lot: 8 80526245. SFM Kit: ATCC Cat: PCS-201-040 Lot: 81209235. FBS: ATCC Cat: 30-2021 Lot: 80609240. Penicillin-Streptomycin: ATCC Cat: 30-230 Lot: 80729231. PBS: Cat: ATCC 30-2200 Lot: 01888. To prepare cell suspension for testing, 100% confluent T75 flask (P4) was harvested using trypsin-EDTA solution. Trypsin was neutralized with culture medium, and cells counted with a hemocytometer. The cells were then seeded in 24- or 48-well plates at 100,000 cells/well density and allowed to reach confluency (approximately 48 or 24 h, respectively). The scratch was established using sterile 200 μL pipette tip or a 3D-printed scratch tool and the wells were gently washed with phosphate buffered saline (PBS) to remove detached cells and debris. At this point, three different positions for each sample were evaluated using ImageJ (open-source image analysis and processing software) for “wound area” at t=0. The treatments were prepared by supplementing serum-free fibroblast medium with varying concentrations of peptides. The plates were incubated at 37° C. and 5% CO₂ for 6 h followed by analysis of the “wound area” using the same methodology as the t=0 data. The wound closure was calculated based on the respective wound areas for each sample at t=0 vs t=6 h and averaged for all 3 individual readouts and all 3 sample replicates. In assay control was hydrolyzed collagen at fixed concentration of 0.25 mg/mL. The data for peptide 4 is shown in FIG. 1 and shows dose-dependent effect of peptide 4 and an overall increase in wound closure by up to 100% over control. Specifically, at 0.25 mg/mL of peptide 4, wound area is 40% closed compared to 20% wound closure for the control (medium with 2% serum).

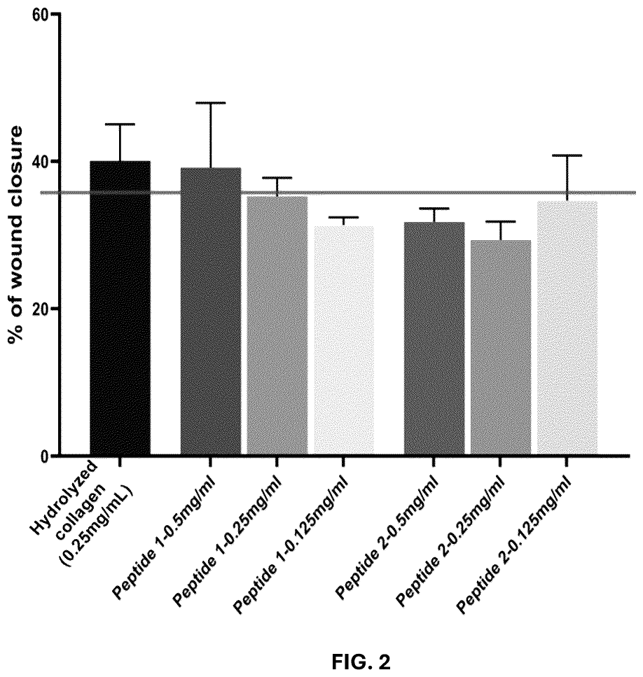

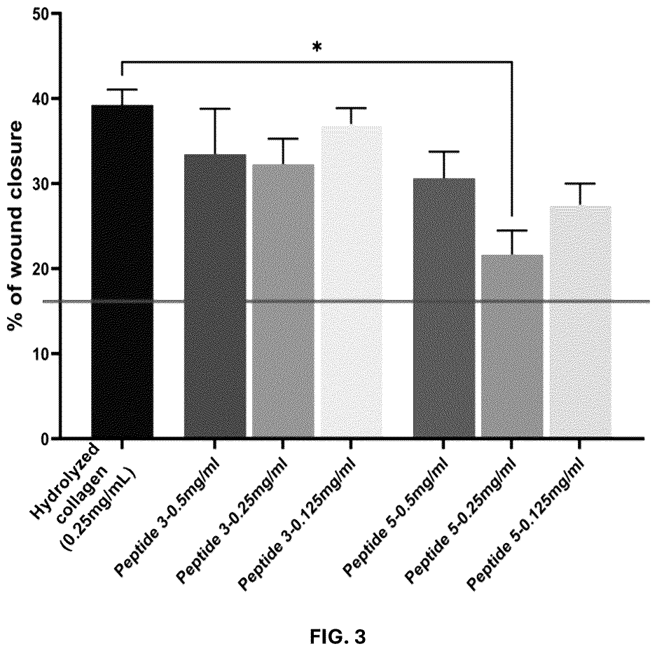

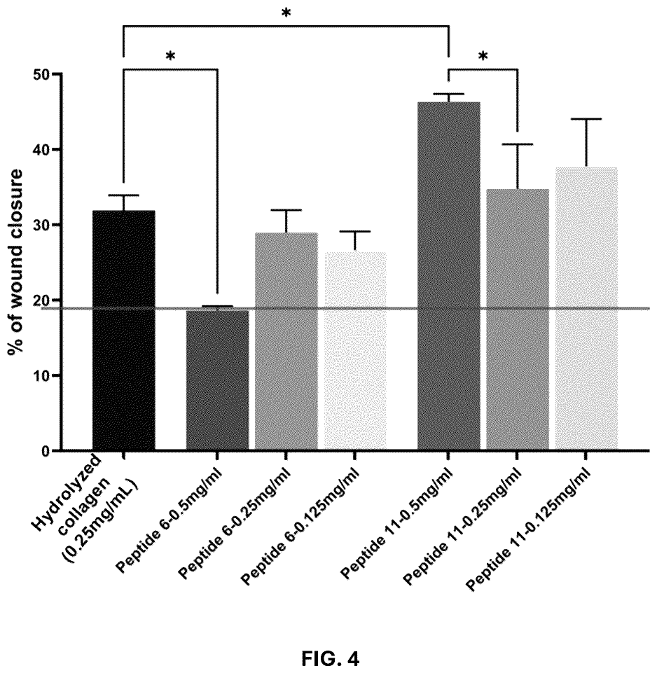

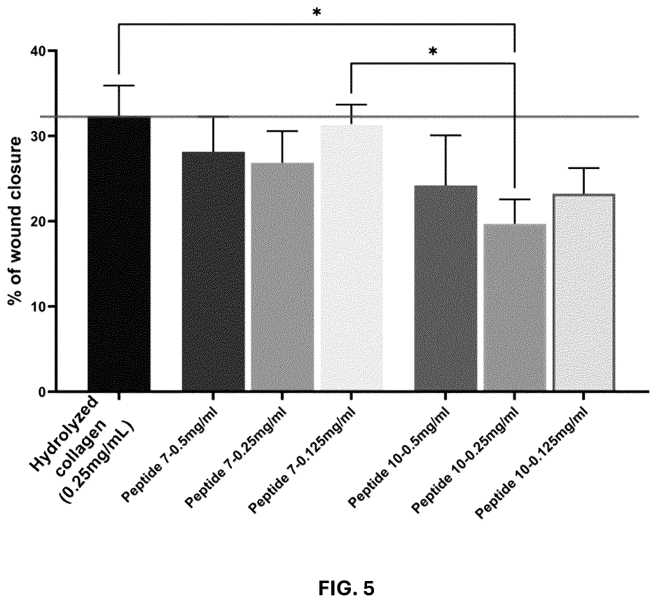

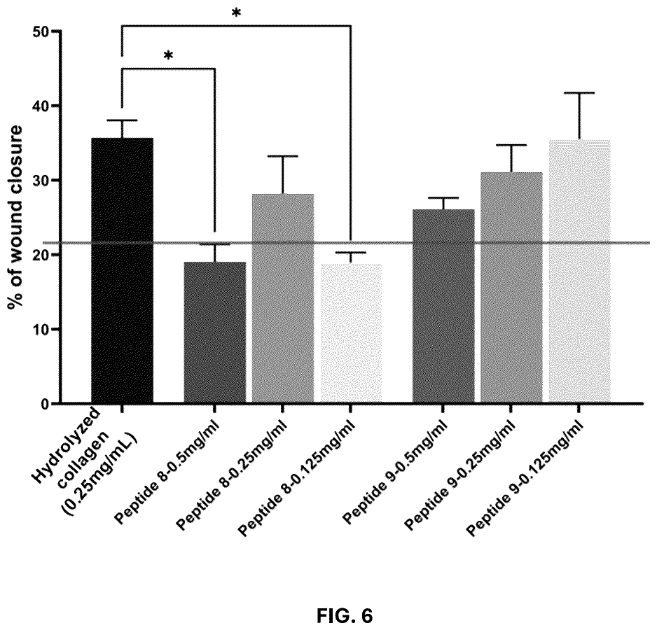

The results for the tested peptides 1-3 and 5-11 are shown in FIGS. 2-6. Of note, besides peptide 4, is peptide 11, which showed marked levels of wound closure. Besides these, peptides 1, 3, 6, 7, and 9 also showed favorable effects. Table 2 lists the summary of the tested peptides and an analysis of their sequence-driven effects.

TABLE 2

| SEQ ID | | Effect on Cell | |
|---|---|---|---|
| No. | Sequence | Migration | Sequence-Driven Effects |
| 1 | GKSGDRGETGPAGPAGPIGPV | Similar to HyCol1 | 2 × R/K (cationic) + 21 aa + Gly-rich |
| 2 | GKSGDRGETGPAGPAGPIGPVGAR | < HyColl | 24 aa, Peptide 1 + C-term GAR |
| | | | terminal R). |
| 3 | GPAGPIGPVGAR | Similar to HyCol1 | Short (12 aa), Gly-Pro rich + C-term |
| | | | GAR (terminal R) |
| 4 | GPIGPVGAR | > HC | Short (9 aa), C-term GAR (terminal R) |
| 5 | GEGGPQGPRGSEGPQGVR | < HyColl | 2 × R + Gly-Pro core (terminal R) |
| 6 | GPRGPAGPSGPAGKDGRIGQPGAVGP | Similar to HyCol1 | 3 × R/K + long (28 aa) + hydrophobic |
| | A | | patches |
| 7 | GAVGPRGPSGPQ | Similar to HyCol1 | Short (12 aa), R |
| 8 | GSDGSVGPVGPA | Below/similar to | Short (12 aa), 2 × V + zero R/K; |
| | | SFM | hydrophobic |
| 9 | GPQGPPGSIGPQ | Similar to HC | Short (12 aa), Gly-Pro rich, no R |
| 10 | GPPGSPGPA | Below/similar to | Short (9 aa), zero R/K |
| | | SFM | |
| 11 | PGTSGPPGLAGPPGSPGPQ | > HyCol1 (Top | Gly-Pro rich (the only one which start |
| | | non-) | with P) + C-term Q (glutamine); zero |
| | | | R/K |

Many in vitro and ex vivo cell and tissue culture models are commercially available for evaluation of individual compounds, mixtures, and formulations with respect to their interaction with cells and tissues and the relevant responses and thus can be employed to exemplify the therapeutic properties of the hydroxyproline-free peptides of this disclosure. In the 2D cell culture models this most commonly includes evaluation of cell proliferation and cell migration, widely applicable to a variety of cell types, which can include or exclude keratinocytes, fibroblasts, and endothelial cells. Additionally, macrophage differentiation and polarization can also be evaluated to identify whether the treatments can modify the course of the wound healing progression cascade. The most promising candidates (individual compounds, mixtures, and formulations) can be further evaluated in the 3D models. The 3D tissue models can include tissue models (e.g., EpiDerm model (website: interactive-mattekcom.pantheonsite.io/mattekproduct/epidermft/) or ex vivo human dermal tissue. These tissues can be evaluated with or without artificial wounding prior to treatment exposure (i.e., surgical wound, burn, etc.), as well as in absence or presence of microbial contamination (simulating infection). Readouts can include tissue properties (i.e., histopathological assessment, tensile strength), biomarker analysis (i.e., pro- and anti-inflammatory cytokines), and expanded to include whole proteome and genome analysis in response to treatments and compared to untreated control. In some embodiments, this disclosure provides for:

A1. A peptide comprising the sequence:

(SEQ ID NO: 12)
X₁X₂X₃X₄X₅X₆X₇X₈X₉GPX₁₀GPX₁₁GX₁₂X₁₃X₁₄X₁₅X₁₆X₁₇X₁₈
X₁₉X₂₀X₂₁X₂₂X₂₃X₂₄X₂₅

wherein X₁, X₂, X₃, X₄, X₅, X₆, X₇, X₈, X₉, X₁₂, X₁₃, X₁₄, X₁₅, X₁₆, X₁₇, X₁₈, X₁₉, X₂₀, X₂₁, X₂₂, X₂₃, X₂₄, and X₂₅ are independently selected from no amino acid, direct bond, G, K, I, S, D, R, E, T, Q, A, V, and P;
wherein X₁₀ is selected from I, R, Q, P, and A;
wherein X₁₁ is selected from V, S, P, R, A, and I;
wherein the peptide is from 9 to 50 amino acids in length; and
wherein the peptide does not include hydroxyproline, or a pharmaceutically acceptable salt thereof.

A2. A peptide comprising a sequence selected from:

(SEQ ID NO: 10)
GPPGSPGPA
or
(SEQ ID NO: 11)
PGTSGPPGLAGPPGSPGPQ;

and
wherein the peptide does not include hydroxyproline, or a pharmaceutically acceptable salt thereof.

A3. The peptide of A1, wherein X₁ is G, X₂ is K, X₃ is S, X₄ is G, X₅ is D, X₆ is R, X₇ is G, X₈ is E, X₉ is T, X₁₀ is A, X₁₁ is A, X₁₂ is P, X₁₃ is I, X₁₄ is G, X₁₅ is P, X₁₆ is V; and X₁₇, X₁₈, X₁₉, X₂₀, X₂₁, X₂₂, X₂₃, X₂₄, and X₂₅ are no amino acid such that the peptide comprises the sequence:

(SEQ ID NO: 1)
GKSGDRGETGPAGPAGPIGPV.

A4. The peptide of A1, wherein X₁ is G, X₂ is K, X₃ is S, X₄ is G, X₅ is D, X₆ is R, X₇ is G, X₈ is E, X₉ is T, X₁₀ is A, X₁₁ is A, X₁₂ is P, X₁₃ is V, X₁₄ is G, X₁₅ is A, X₁₆ is R; and X₁₇, X₁₈, X₁₉, X₂₀, X₂₁, X₂₂, X₂₃, X₂₄, and X₂₅ are no amino acid such that the peptide comprises the sequence:

(SEQ ID NO: 2)
GKSGDRGETGPAGPAGPIGPVGAR.

A5. The peptide of A1, wherein X₁, X₂, X₃, X₄, X₅, X₆, X₇, X₈, and X₉ are no amino acid, X₁₀ is A, X₁₁ is I, X₁₂ is P, X₁₃ is V, X₁₄ is G, X₁₅ is A, X₁₆ is R; and X₁₇, X₁₈, X₁₉, X₂₀, X₂₁, X₂₂, X₂₃, X₂₄, and X₂₅ are no amino acid such that the peptide comprises the sequence:

(SEQ ID NO: 3)
GPAGPIGPVGAR.

A6. The peptide of A1, wherein X₁, X₂, X₃, X₄, X₅, X₆, X₇, X₈, X₉, X₁₄, X₁₅, X₁₆, X₁₇, X₁₈, X₁₉, X₂₀, X₂₁, X₂₂, X₂₃, X₂₄, and X₂₅ are no amino acid, X₁₀ is I, X₁₁ is V, X₁₂ is A, and X₁₃ is R such that the peptide comprises the sequence:

(SEQ ID NO: 4)
GPIGPVGAR.

A7. The peptide of A1, wherein X₁, X₂, X₃, X₄, X₅, X₆, are no amino acid, X₇ is G, X₈ is E, X₉ is G, X₁₀ is O, X₁₁ is R, X₁₂ is S, X₁₃ is E, X₁₄ is G, X₁₅ is P, X₁₆ is Q, X₁₇ is V, X₁₈ is R and X₁₉, X₂₀, X₂₁, X₂₂, X₂₃, X₂₄, and X₂₅ are no amino acid such that the peptide comprises the sequence:

(SEQ ID NO: 5)
GEGGPQGPRGSEGPQGVR.

A8. The peptide of A1, wherein X₁, X₂, and X₃ are no amino acid; X₄ is G, X₅ is P, X₆ is R, X₇ is G, X₈ is P, X₉ is A, X₁₀ is S, X₁₁ is A, X₁₂ is K, X₁₃ is D, X₁₄ is G, X₁₅ is R, X₁₆ is I, X₁₇ is G, X₁₈ is Q, X₁₉ is P, X₂₀ is G, X₂₁ is A, X₂₂ is V, X₂₃ is G, X₂₄ is P, and X₂₅ is A such that the peptide comprises the sequence:

(SEQ ID NO: 6)
GPRGPAGPSGPAGKDGRIGQPGAVGPA.

A9. The peptide of A1, wherein X₁, X₂, X₃, X₄, X₅, X₆, are no amino acid, X₇ is G, X₈ is A, X₉ is V, X₁₀ is R, X₁₁ is S, X₁₂ is P, X₁₃ is Q, and X₁₄, X₁₅, X₁₆, X₁₇, X₁₈, X₁₉, X₂₀, X₂₁, X₂₂, X₂₃, X₂₄, and X₂₅ are no amino acid such that the peptide comprises the sequence:

(SEQ ID NO: 7)
GAVGPRGPSGPQ.

A10. The peptide of A1, wherein X₁, X₂, X₃, X₄, X₅, X₆, are no amino acid, X₇ is G, X₈ is A, X₉ is V, X₁₀ is R, X₁₁ is S, X₁₂ is P, X₁₃ is Q, X₁₄, X₁₅, X₁₆, X₁₇, X₁₈, X₁₉, X₂₀, X₂₁, X₂₂, X₂₃, X₂₄, and X₂₅ are no amino acid such that the peptide comprises the sequence:

(SEQ ID NO: 8)
GSDGSVGPVGPA.

A11. The peptide of A1, wherein X₁, X₂, X₃, X₄, X₅, X₆, X₇, X₈, and X₉ are no amino acid, X₁₀ is Q, X₁₁ is P, X₁₂ is S, X₁₃ is I, X₁₄ is G, X₁₅ is P, X₁₆ is Q, X₁₇, X₁₈, X₁₉, X₂₀, X₂₁, X₂₂, X₂₃, X₂₄, and X₂₅ are no amino acid such that the peptide comprises the sequence:

(SEQ ID NO: 9)
GPQGPPGSIGPQ.

A12. The peptide of A1 or A2, wherein the peptide has a C-terminus modification selected from: PEG amidification, amidation, Glycosyl phosphatidylinositol (GPI), detyrosination, N-alkyl amidification, or esterification with a C₄-C₂₂ fatty acid.

A13. The peptide of A1 or A2, wherein the peptide has a N-terminus modification selected from: fatty acid amidification, urea modification, acetylation, carbamylation, formylation, glycation, methylation, myristoylation, and sulfonamidification,

A14. The peptide of A1 or A2, wherein the peptide comprises S, T, or Y, and one or more of the S, T, or Y amino acids in the peptide are phosphorylated.

A15. The peptide of A1 or A2, wherein the peptide comprises T and one or more of the T amino acids in the peptide are sulfated.

A16. The peptide of A1 or A2, wherein the peptide comprises C and one or more of the C amino acids in the peptide are prenylated or geranylgeranylated.

A17. The peptide of A1 or A2, wherein each of the amino acids in the peptide are independently substituted with an unnatural amino acid selected from: citrulline, paphtylanine, alpha-methyl amino acid, d-amino acid, homo-amino acid, selenocysteine, pyrrolysine, ornithine, norleucine, norvaline, 2-amino-heptanoic acid, alpha-aminoisobutyric acid, 2-amino-3-cyclopropylpropanoic acid, cyclohexyl-alanine, alpha-methyl-valine, homo-cysteine, penicillamine, statine, 3-aminobenzoic acid, homoe-phenyl-alanine, 4-fluorophenyl-alanine, 5-fluoro-tryptophan, 3-pyridyl-alanine, 3-nitrotyrosine, nitroarginine, pyroglutamic acid, tert-leucine, p-(propargyloxy)phenylalanine, p-methoxyphenylalanine, dansylalanine, DMNB-serine, O-methyl-L-tyrosine, an L-3-(2-naphthyl) alanine, an O-4-allyl-L-tyrosine, an O-propargyl-L-tyrosine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, 3-nitro-L-tyrosine, 4-nitro-L-phenylalanine, 3-amino-L-tyrosine, p-carboxymethyl-Lphenylalanine, biphenyl-alanine, bipyridyl-alanine, 1,5-dansyl-alanine, o-nitrobenzyl-serine, p-cyano-L-phenylalanine, m-cyano-L-phenylalanine, p-ethylthiocarbonyl-L-phenylalanine, p-isopropylthiocarbonyl-L-phenylalanine, 7-amino-coumarine-alanine, and 7-hydroxy-coumarin alanine.

A18. The peptide of A1 or A2, wherein the peptide is connected at the C-terminus, the N-terminus, or both ends, to a tag sequence selected from FLAG (DYKDDDDK) (SEQ ID NO: 31), HA (YPYDVPDYA) (SEQ ID NO: 32), HA12CA5 (CYPYDVPDYA) (SEQ ID NO: 33), Myc (EQKLISEEDL) (SEQ ID NO: 34), His (HHHHHH) (SEQ ID NO: 35), V5 (GKPIPNPLLGLDST) (SEQ ID NO: 36), or RGD (RGD).

A19. The peptide of A1 or A2, wherein the peptide is cyclized.

A20. The peptide of A19, wherein the cyclized peptide is through the backbone via an amide bond, by cysteine-cysteine crosslinks, by thio-ether cyclization, by lactam formation, click chemistry with Cu(I)-catalyzed azide-alkynes and strain-promoted azide-alkynes, ligation between tetrazine and alkene, bi-functional PEG linkers, and thiol-ene chemistry or combinations thereof.

A21. The peptide of A1 or A2, wherein the peptide is connected at the C-terminus, the N-terminus, or both ends, to a cell-penetrating peptide.

A22. The peptide of A1 or A2, wherein the peptide is connected at the C-terminus, the N-terminus, or both ends, to a cleavable enzyme substrate sequence.

A23. The peptide of A22, wherein the cleavable enzyme substrate sequence is of a mammalian or microbial protease present in a wound infection.

A24. The peptide of A1 or A2, wherein the peptide is connected at the C-terminus, the N-terminus, or both ends, to a glycopeptide, protein, glycoprotein, glycosaminoglycan, or nucleic acid.

A25. The peptide of A24, wherein the glycopeptide, protein, glycoprotein, glycosaminoglycan is selected from: collagen, gelatin, or polysaccharides.

A26. The peptide of A1 or A2, wherein the peptide is covalently or non-covalently anchored to a biodegradable scaffold.

A27. The peptide of A1, wherein the peptide consists of a peptide of any of SEQ ID Nos. 1-9.

A28. The peptide of A2, wherein the peptide consists of a peptide of any of SEQ ID Nos. 10-11.

A29. A composition comprising one or more peptides of A1.

A30. A composition comprising one or more peptides of A1, and a pharmaceutically acceptable carrier.

A31. A composition comprising one or more peptides of A2.

A32. A composition comprising one or more peptides of A2, and a pharmaceutically acceptable carrier.

A33. The composition of A29 or 31, further comprising a mammalian cell.

A34. The composition of A33, wherein the cells are selected from autologous or allogeneic human stem cells.

A35. The composition of A29 or 31, further comprising a biologically active agent selected from: micronized tissue, morselized tissue, minced tissue, granulated crosslinked bovine tendon collagen, hydrolyzed collagen, amniotic fluid, Wharton's Jelly, or an antimicrobial agent.

A36. The composition of A35, wherein the biologically active agent is hydrolyzed collagen and the peptide of A1 or A2 is enriched relative to its natural abundance in hydrolyzed collagen.

A37. A scaffold comprising a peptide of A1 or A2 and a structural support agent selected from: collagen, gelatin, glycosaminoglycans, glycoproteins, polysaccharides, polyvinylpyrrolidone, poly(lactic-co-glycolic) acid, polyvinyl alcohol, poly(lactide-co-caprolactone), polycaprolactone, polyhydroxybutyrate, polylactic acid, polyglycolic acid and combinations thereof; silicones, polyolefins, acrylic resins, polyurethanes and combinations thereof.

A38. The scaffold of A35, wherein the scaffold has been formed into fibers by a method selected from electrospinning, centrifugal spinning (force spinning), melt blowing, phase separation, self-assembly, template synthesis, drawing, extrusion, interfacial polymerization, and melt-spinning.

A39. A method of treating compromised tissue, comprising contacting injured or compromised tissue with a treatment composition of A29 or A31.

A40. The method of A39, wherein the treatment composition further comprises biologic components such as cells, minced tissue, reactive proteins, polysaccharides, and biologic fluids.

A41. The method of A37, wherein the contacting step comprises applying the treatment composition within tissue.

A42. The method of A3, wherein the contacting step comprises topical application of the treatment composition.

A43. The method of A39, wherein the treatment composition comprises the composition of A29 or A31 in a liquid carrier.

A44. The method of A39, wherein the compromised tissue is at least one of a cut, a wound, a lesion, a fracture, a tear, a break, a rash, a fistula, a burn, a void, a surgical site, an ulcer, or a medical implant site.

A45. A method of modulating the migration of a fibroblast, keratinocyte, endothelial cell, macrophage, or pericyte present in or near the skin, the method comprising contacting the fibroblast, keratinocyte, endothelial cell, macrophage, or pericyte, respectively, with a peptide of A1 or A2.

A46. The use of a peptide of A1 or A2 in the manufacture of a medicament for therapeutically treating compromised tissue in a subject.

A47. A peptide of A1 or A2 for use in treating compromised tissue in a subject.

All publications, patents and patent applications cited herein are hereby incorporated by reference in their entirety.

While the above specification contains many specifics, these should not be construed as limitations on the scope of the invention, but rather as examples of preferred embodiments thereof. Many other variations are possible. Accordingly, the scope of this invention should be determined not by the embodiments illustrated, but by the appended claims and their legal equivalents.

We claim:

1. A peptide comprising the sequence:
(SEQ ID NO: 12)
X₁X₂X₃X₄X₅X₆X₇X₈X₉GPX₁₀GPX₁₁GX₁₂X₁₃X₁₄X₁₅X₁₆X₁₇X₁₈X₁₉X₂₀X₂₁X₂₂
X₂₃X₂₄X₂₅
wherein X₁, X₂, X₃, X₄, X₅, X₆, X₇, X₈, X₉, X₁₂, X₁₃, X₁₄, X₁₅, X₁₆, X₁₇, X₁₈, X₁₉, X₂₀, X₂₁, X₂₂, X₂₃, X₂₄, and X₂₅ are independently selected from no amino acid, direct bond, G, K, I, S, D, R, E, T, Q, A, V, and P;
wherein X₁₀ is selected from I, R, Q, P, and A;
wherein X₁₁ is selected from V, S, P, R, A, and I;
wherein the peptide is from 9 to 50 amino acids in length; and
wherein the peptide does not include hydroxyproline, or a pharmaceutically acceptable salt thereof.

2. A peptide comprising a sequence selected from:
```
  (SEQ ID NO: 10)
GPPGSPGPA
or
  (SEQ ID NO: 11)
PGTSGPPGLAGPPGSPGPQ;
and
```
wherein the peptide does not include hydroxyproline, or a pharmaceutically acceptable salt thereof.

3. The peptide of claim 1, wherein the peptide is one of the following:
```
  (SEQ ID NO: 1)
GKSGDRGETGPAGPAGPIGPV,
(SEQ ID NO: 2)
GKSGDRGETGPAGPAGPIGPVGAR,
  (SEQ ID NO: 3)
GPAGPIGPVGAR,
  (SEQ ID NO: 4)
GPIGPVGAR,
  (SEQ ID NO: 5)
GEGGPQGPRGSEGPQGVR,
(SEQ ID NO: 6)
GPRGPAGPSGPAGKDGRIGQPGAVGPA,
  (SEQ ID NO: 7)
GAVGPRGPSGPQ,
  (SEQ ID NO: 8)
GSDGSVGPVGPA,
or
  (SEQ ID NO: 9)
GPQGPPGSIGPQ.
```

4. The peptide of claim 1, wherein the peptide has a C-terminus modification selected from: PEG amidification, amidation, Glycosyl phosphatidylinositol (GPI), detyrosination, N-alkyl amidification, or esterification with a C₄-C₂₂ fatty acid, or a N-terminus modification selected from: fatty acid amidification, urea modification, acetylation, carbamylation, formylation, glycation, methylation, myristoylation, and sulfonamidification.

5. The peptide of claim 1, wherein the peptide comprises S, T, or Y, and one or more of the S, T, or Y amino acids in the peptide are phosphorylated.

6. The peptide of claim 1, wherein the peptide comprises T and one or more of the T amino acids in the peptide are sulfated.

7. The peptide of claim 1, wherein the peptide comprises C and one or more of the C amino acids in the peptide are prenylated or geranylgeranylated.

8. The peptide of claim 1, wherein from one to four of the amino acids in the peptide are independently substituted with an unnatural amino acid selected from: citrulline, paphtylanine, alpha-methyl amino acid, d-amino acid, homo-amino acid, selenocysteine, pyrrolysine, ornithine, norleucine, norvaline, 2-amino-heptanoic acid, alpha-aminoisobutyric acid, 2-amino-3-cyclopropylpropanoic acid, cyclohexyl-alanine, alpha-methyl-valine, homo-cysteine, penicillamine, statine, 3-aminobenzoic acid, homoe-phenyl-alanine, 4-fluorophenyl-alanine, 5-fluoro-tryptophan, 3-pyridyl-alanine, 3-nitrotyrosine, nitroarginine, pyroglutamic acid, tert-leucine, p-(propargyloxy)phenylalanine, p-methoxyphenylalanine, dansylalanine, DMNB-serine, O-methyl-L-tyrosine, an L-3-(2-naphthyl) alanine, an O-4-allyl-L-tyrosine, an O-propargyl-L-tyrosine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, 3-nitro-L-tyrosine, 4-nitro-L-phenylalanine, 3-amino-L-tyrosine, p-carboxymethyl-Lphenylalanine, biphenyl-alanine, bipyridyl-alanine, 1,5-dansyl-alanine, o-nitrobenzyl-serine, p-cyano-L-phenylalanine, m-cyano-L-phenylalanine, p-ethylthiocarbonyl-L-phenylalanine, p-isopropylthiocarbonyl-L-phenylalanine, 7-amino-coumarine-alanine, and 7-hydroxy-coumarin alanine.

9. The peptide of claim 1, wherein the peptide is connected at the C-terminus, the N-terminus, or both ends, to a tag sequence selected from FLAG (DYKDDDDK) (SEQ ID NO: 31), HA (YPYDVPDYA) (SEQ ID NO: 32), HA12CA5 (CYPYDVPDYA) (SEQ ID NO: 33), Myc (EQKLISEEDL) (SEQ ID NO: 34), His (HHHHHH) (SEQ ID NO: 35), V5 (GKPIPNPLLGLDST) (SEQ ID NO: 36), or RGD (RGD); a cell-penetrating peptide; a cleavable enzyme substrate sequence; a glycopeptide; a protein; a glycoprotein; a glycosaminoglycan; a nucleic acid; or a biodegradable scaffold.

10. The peptide of claim 1, wherein the peptide is cyclized.

11. The peptide of claim 1, wherein the peptide consists of a peptide of any of SEQ ID Nos. 1-9.

12. The peptide of claim 2, wherein the peptide consists of a peptide of any of SEQ ID Nos. 10-11.

13. A composition comprising one or more peptides of claim 1.

14. The composition of claim 13, further comprising a pharmaceutically acceptable carrier.

15. A composition comprising one or more peptides of claim 2.

16. The composition of claim 15, further comprising a pharmaceutically acceptable carrier.

17. The composition of claim 13, further comprising a mammalian cell.

18. The composition of claim 13, further comprising a biologically active agent selected from: micronized tissue, morselized tissue, minced tissue, granulated crosslinked bovine tendon collagen, hydrolyzed collagen, amniotic fluid, Wharton's Jelly, or an antimicrobial agent.

19. A scaffold comprising a peptide of claim 1 and a structural support agent selected from: collagen, gelatin, glycosaminoglycans, glycoproteins, polysaccharides, polyvinylpyrrolidone, poly(lactic-co-glycolic) acid, polyvinyl alcohol, poly(lactide-co-caprolactone), polycaprolactone, polyhydroxybutyrate, polylactic acid, polyglycolic acid and combinations thereof; silicones, polyolefins, acrylic resins, polyurethanes and combinations thereof.

20. The method of claim 19, wherein the compromised tissue is at least one of a cut, a wound, a lesion, a fracture, a tear, a break, a rash, a fistula, a burn, a void, a surgical site, an ulcer, or a medical implant site.


SEQUENCE LISTING (36 sequences)

SEQ ID NO: 1 - protein, 21 aa, synthetic construct
GKSGDRGETGPAGPAGPIGPV

SEQ ID NO: 2 - protein, 24 aa, synthetic construct
GKSGDRGETGPAGPAGPIGPVGAR

SEQ ID NO: 3 - protein, 12 aa, synthetic construct
GPAGPIGPVGAR

SEQ ID NO: 4 - protein, 9 aa, synthetic construct
GPIGPVGAR

SEQ ID NO: 5 - protein, 18 aa, synthetic construct
GEGGPQGPRGSEGPQGVR

SEQ ID NO: 6 - protein, 27 aa, synthetic construct
GPRGPAGPSGPAGKDGRIGQPGAVGPA

SEQ ID NO: 7 - protein, 12 aa, synthetic construct
GAVGPRGPSGPQ

SEQ ID NO: 8 - protein, 12 aa, synthetic construct
GSDGSVGPVGPA

SEQ ID NO: 9 - protein, 12 aa, synthetic construct
GPQGPPGSIGPQ

SEQ ID NO: 10 - protein, 9 aa, synthetic construct
GPPGSPGPA

SEQ ID NO: 11 - protein, 19 aa, synthetic construct
PGTSGPPGLAGPPGSPGPQ

SEQ ID NO: 12 - protein, 30 aa, synthetic construct
  VARIANT 1..9: note = no amino acid, direct bond, G, K, I, S, D, R, E, T, Q, A, V, or P
  VARIANT 12..25: note = no amino acid, direct bond, G, K, I, S, D, R, E, T, Q, A, V, or P
  VARIANT 10: note = I, R, Q, P, or A
  VARIANT 11: note = V, S, P, R, A, or I
XXXXXXXXXGPXGPXGXXXXXXXXXXXXXX

SEQ ID NO: 13 - protein, 16 aa, synthetic construct
RQPKIWFPNRRKPWKK

SEQ ID NO: 14 - protein, 11 aa, synthetic construct
GRKKRRQRPPQ

SEQ ID NO: 15 - protein, 26 aa, synthetic construct
GWTLNSAGYLLGKINKALAALAKKIL

SEQ ID NO: 16 - protein, 21 aa, synthetic construct
TRSSRAGLQFPVGRVHRLLRK

SEQ ID NO: 17 - protein, 9 aa, synthetic construct
RKKRRQRRR

SEQ ID NO: 18 - protein, 16 aa, synthetic construct
RQIKIWFQNRRMKWKK

SEQ ID NO: 19 - protein, 18 aa, synthetic construct
KLALKLALKALKAALKLA

SEQ ID NO: 20 - protein, 16 aa, synthetic construct
AAVALLPAVLLALLAP

SEQ ID NO: 21 - protein, 10 aa, synthetic construct
VPMLKPMLKE

SEQ ID NO: 22 - protein, 28 aa, synthetic construct
MANLGYWLLALFVTMWTDVGLCKKRPKP

SEQ ID NO: 23 - protein, 18 aa, synthetic construct
LLIILRRRIRKQAHAHSK

SEQ ID NO: 24 - protein, 21 aa, synthetic construct
KETWWETWWTEWSQPKKKRRV

SEQ ID NO: 25 - protein, 18 aa, synthetic construct
RGGRLSYSRRRFSTSTGR

SEQ ID NO: 26 - protein, 15 aa, synthetic construct
SDLWEMMMVSLACQY

SEQ ID NO: 27 - protein, 12 aa, synthetic construct
TSPLNIHNGQKL

SEQ ID NO: 28 - protein, 16 aa, synthetic construct
RVIRVWFQNKRCKDKK

SEQ ID NO: 29 - protein, 27 aa, synthetic construct
GALFLGFLGAAGSTMGAWSQPKKKRKV

SEQ ID NO: 30 - protein, 27 aa, synthetic construct
GALFLAFLAAALSLMGLWSQPKKKRRV

SEQ ID NO: 31 - protein, 8 aa, synthetic construct
DYKDDDDK

SEQ ID NO: 32 - protein, 9 aa, synthetic construct
YPYDVPDYA

SEQ ID NO: 33 - protein, 10 aa, synthetic construct
CYPYDVPDYA

SEQ ID NO: 34 - protein, 10 aa, synthetic construct
EQKLISEEDL

SEQ ID NO: 35 - protein, 6 aa, synthetic construct
HHHHHH

SEQ ID NO: 36 - protein, 14 aa, synthetic construct
GKPIPNPLLGLDST